SEQUENCE LISTING (92 sequences)

SEQ ID NO: 1 - DNA, 90 bp, Bacillus anthracis
accttttgctaaagtgatttcaccttttgcattaattttttctgcagctggcgttacaaattcaataccctctggtgaaacaacttcagt

SEQ ID NO: 2 - DNA, 157 bp, Bacillus anthracis
caaagtggcaaaaggggaatttagaatatgctagtttccgttctatgacaatcgatgaatttattgatccgttgtcttttcttaaaaagattgcttctttgtttgaattagaaattcaatatcgtgctggagttgtaggctctcaagttgttgggcg

SEQ ID NO: 3 - DNA, 155 bp, Bacillus anthracis
tcccaatacatatgagcgattcgcctttataaacgacgtattcctttgaactcgttatgacactcattactcaactccccttttctactaaaatagcgtttttgtttggtttttttcttcacataatccgtcctatttgatttttacataccacc

SEQ ID NO: 4 - DNA, 160 bp, Bacillus anthracis
ttggatcagcgtttctgaattcagctaataatacaacgtcttctggatctaatgaatataatcttgttaatgcaagtggagttgatataaacgtagcacagaaacttaggggaatacgtaattatagcattaaagtagatggaattgagcgatatgggga

SEQ ID NO: 5 - DNA, 151 bp, Bacillus anthracis
ggccccataacttgatttctcttcaatatgaaagttgctgcctcatcaacattttcgaaaataaaatcttcatcataaatcgtatgacaaccttcccaccttataattataggaaaggcacccgacgccattccttcagctggagcaagat

SEQ ID NO: 6 - DNA, 135 bp, Bacillus anthracis
gaaatgaatgcagaaaaattagagcttgcgaaaaatccacctgacagtgcagcagtacaagaaatatttgctcaattagaaggttggggtaaagctattgctaaaggtggagaagaaggtaaacaagcatttgta

SEQ ID NO: 7 - DNA, 156 bp, Bacillus anthracis
aattctcatcctacagcatctccaataatatatattccaaccaaaagatgaccatgttacggttataaaataccaatctattatttcatcgcgtccttcacaacatcttgaatcataacgacttctgttttcatatcaacagagggtgcttcttca

SEQ ID NO: 8 - DNA, 146 bp, Bacillus anthracis
ggtgggtaaatgatgagtgtttcaacaaaaattaatcagctaataagtcttagagaaaaaatcgctaaagatttagggttttatgttaactttgatttagcgaattcaagttggtcatactctaaaaaggctggtcaaatcaaaaa

SEQ ID NO: 9 - DNA, 111 bp, Bacillus anthracis
tgtggaagaatccactgtccaagaacgtgagcgtgtgctatgggatgaaatggcttcaccaataggatggactcctgttactgggcaagttgaggaaatgaaagagacaga

SEQ ID NO: 10 - DNA, 164 bp, Bacillus anthracis
gaaggacgatacagacatttattgggaactacacatgcactagggtcattgttcattagctcatcgattgcattattaacaacggcttttattgtccttacgcatttatctagctttatgaacaattactatttttggatagggcttgctattcaacttgcggt

SEQ ID NO: 11 - DNA, 102 bp, Bacillus anthracis
tcgcaagataaatgggaagacgaaaagcgggctggacaatattctccgacataccagggacagcttccaagtgaaatggtaaatgtgattggtacgggagga

SEQ ID NO: 12 - DNA, 135 bp, Bacillus anthracis
ttggtaaagtagaagcatcttggttaagagcaggattactcatgggtatgacgataaaaacaagcaatacaaatgaacatttgcatatggaaaaccaggtgatgcgttttgtaaaccaagggtcagataagatgg

SEQ ID NO: 13 - DNA, 138 bp, Bacillus anthracis
atatgtggttaggagccaatgaaggtgatggtgacttctttgtagataaacatactttacaaaaaagcttgtacaagccagcttctgattgggaatggtggagaggaaaacgtcaggtgattattggactcgatttgt

SEQ ID NO: 14 - DNA, 152 bp, Bacillus anthracis
actactcgacaaggacgtttgtatttccggctgtcagtgtcggcgcgccgcatcaaagataccggacatttattgttggccactccaacgacaagtcaaaattacaaaccgatccgagagttgtgcccttcagaagcaaatgggaaacatgg

SEQ ID NO: 15 - DNA, 134 bp, Bacillus anthracis
tgatatttatgaccaagattcaatatacgaatataatcccaaaagagccaatcatgcaatggagttcattgagaatttttgcaagcattcaaaggggaaatgggctggaaaacctattgaccttgagctatggc

SEQ ID NO: 16 - DNA, 179 bp, Bacillus anthracis
ccaggtccatcctatgttgcagtagaaacgtatgttacagtagaagcgtattttagcaaacgtgagtacgcactacctccagctagatactatcatcaagaaacgcgagatgggatgatatattctggttaccttaccagacatagttatgcatcaacgggagacacgcggattgtagt

SEQ ID NO: 17 - DNA, 244 bp, Bacillus anthracis
cacgtgagtttacaccatacgcggcgaacgcaaagcgttcaagattagaacaagaagtgaatattgatttttataaaagagaaattttcacatatgcagatgcttgtatcgtcacagtaaggattacaaatccagatggctcaactgagtatcaaaaaggagaaataagtacagaaaacattgtatgtactaatatcgtatggggcgaggatgaagtttcattcgaaatgagagcaagtgctag

SEQ ID NO: 18 - DNA, 147 bp, Bacillus anthracis
ggaacaactttcccacaagctgaacttgagtagaagccagatcaaaaactgggaaactgatcggtatcagccagatatagatacgttggttatcatcgcctccttcttcaatgtttcggtagacgcgcttattgggttcacgaatga

SEQ ID NO: 19 - DNA, 168 bp, Bacillus anthracis
aggaacgtgcgacgactagctgaaaagcgtagggtgtaagccaatgacatccgaaatggggagcatcttatataaaagatgatgatatagtctggtctgtatagtgatatacagaagttcataagagaactggcaggatgttgcgagtcctgttgaacatatcgggtg

SEQ ID NO: 20 - DNA, 212 bp, Bacillus anthracis
tttcttatccaacaggtgtatgcttatgttttataaaaaagtttgatcatagcttatttacaattctttgattaaagagataaagcatcgcaccttttttataaacgtcttaactttatttcaaaactacactatatttactaacctactaatctcctactcaaccgtaacagacttagctaagttacgtggcttatcaacgtcacactcgc

SEQ ID NO: 21 - DNA, 167 bp, Bacillus anthracis
tcaaacgttgccgaataagccaattcatttagatgcgttacttaataaactatttttaggtaatacgaaggaccgtattaaagcagcaattgagttaattgagtgggtgcttcataaataatattgtctaatattgtcggtaaatcgatatgttgtgtcgaatcgtc

SEQ ID NO: 22 - DNA, 205 bp, Bacillus anthracis
ttggcattgtgttagatgaggggtatttctatgatgaattaacattgaaagaaatgaaaaatgtgattgccccttcttatactgactgggatgaagcagtcttcttatcttatattaaacaatttaatttaaatctaagacaaaaaatttctactctatctaaagggatgcggatgaaatttgcggttgctttggctttatcaca

SEQ ID NO: 23 - DNA, 167 bp, Bacillus anthracis
cagagcgtttcttaaatgaagaggatttaattggagagcatttagagagagaagatattgatatcacatatttagatgacacgatgtatatattgttgccatataagaatgatcaagaattaaaacagaaaaagttagataaaatccaaattgaggcatatgatgaa

SEQ ID NO: 24 - DNA, 242 bp, Bacillus anthracis
aaaggcggtttagaatttggaccgtatcaaaatttaaaacaagttgagtcgatagcagaaaagccaattcaagcatttcaagttttaaataccattcttgagaaaatggaagagaaaatgttctatatgaaggaaagacattatacaaacgttgtagaaacaaatataaaagagcgtcatttcattatagttgatgaaggggctgaactttgtcctgataaaagcatgggtaaagagcagca

SEQ ID NO: 25 - DNA, 197 bp, Bacillus anthracis
tgtaaatgaacgccttgaccaatcaccagcagggaattcagtttgtagattttctgtccatgatccgttgataaattggaatacaagaaaagtaacgtaaacatcattttgaagatcgacactgaatgtcatgtctttacctttttcaaaattacccatcttagtattatctaaatggaattgatagaaggagtcgg

SEQ ID NO: 26 - DNA, 145 bp, Bacillus anthracis
aaacaatgttggatggtaaatggcattacttccacggcaaattaaaagttgtggatgggaaagtgcgtgtttactttgggatgttgaagacgggaaatgtaaaaggtgctaaaacaagaataaccggagtgaaatttgcgaatgg

SEQ ID NO: 27 - DNA, 237 bp, Bacillus anthracis
ccgagctttaatggaaatggagaattattatatgattttagggatttcatattttaatattttttgtattgtagtgattatcttgtttttctttattttttggagattgcatgtatggtataacaagaaacacaatgttcctaaggtttttcaatggtttcctagaaaatggggtaaaagaaaagtgtcagagcatttatcacaattgaatgaaaagctagaggctgaaggtaaagg

SEQ ID NO: 28 - DNA, 134 bp, Bacillus anthracis
ggagaaacagatagtggtgaaagctgggagtatatttacttcttagatgaaataaagcatcaagccattagtttaagtgtctttaatagattattagattatgaagaaggaaatctaatacaaggctttagagt

SEQ ID NO: 29 - DNA, 20 bp, Artificial Sequence
  Description of Artificial Sequence Synthetic primer
gtagactgcgtaccaattcc

SEQ ID NO: 30 - DNA, 20 bp, Artificial Sequence
  Description of Artificial Sequence Synthetic primer
gacgatgagtcctgagtaag

SEQ ID NO: 31 - DNA, 12 bp, Artificial Sequence
  Description of Artificial Sequence Synthetic oligonucleotide
taacctgcccgg

SEQ ID NO: 32 - DNA, 12 bp, Artificial Sequence
  Description of Artificial Sequence Synthetic oligonucleotide
taacctcggccg

SEQ ID NO: 33 - DNA, 18 bp, Artificial Sequence
  Description of Artificial Sequence Synthetic primer
tgtaaaacgacggccagt

SEQ ID NO: 34 - DNA, 18 bp, Artificial Sequence
  Description of Artificial Sequence Synthetic primer
caggaaacagctatgacc

SEQ ID NO: 35 - DNA, 24 bp, Artificial Sequence
  Description of Artificial Sequence Synthetic primer
ctaccttaggaccgttatagttac

SEQ ID NO: 36 - DNA, 21 bp, Artificial Sequence
  Description of Artificial Sequence Synthetic primer
aggtaggcgaggagagaatcc

SEQ ID NO: 37 - DNA, 29 bp, Artificial Sequence
  Description of Artificial Sequence Synthetic primer
accttttgctaaagtgatttcaccttttg

SEQ ID NO: 38 - DNA, 29 bp, Artificial Sequence
  Description of Artificial Sequence Synthetic primer
actgaagttgtttcaccagagggtattga

SEQ ID NO: 39 - DNA, 25 bp, Artificial Sequence
  Description of Artificial Sequence Synthetic primer
caaagtggcaaaaggggaatttaga

SEQ ID NO: 40 - DNA, 25 bp, Artificial Sequence
  Description of Artificial Sequence Synthetic primer
cgcccaacaacttgagagcctacaa

SEQ ID NO: 41 - DNA, 25 bp, Artificial Sequence
  Description of Artificial Sequence Synthetic primer
tcccaatacatatgagcgattcgcc

SEQ ID NO: 42 - DNA, 29 bp, Artificial Sequence
  Description of Artificial Sequence Synthetic primer
ggtggtatgtaaaaatcaaataggacgga

SEQ ID NO: 43 - DNA, 25 bp, Artificial Sequence
  Description of Artificial Sequence Synthetic primer
ttggatcagcgtttctgaattcagc

SEQ ID NO: 44 - DNA, 25 bp, Artificial Sequence
  Description of Artificial Sequence Synthetic primer
tccccatatcgctcaattccatcta

SEQ ID NO: 45 - DNA, 25 bp, Artificial Sequence
  Description of Artificial Sequence Synthetic primer
ggccccataacttgatttctcttca

SEQ ID NO: 46 - DNA, 25 bp, Artificial Sequence
  Description of Artificial Sequence Synthetic primer
atcttgctccagctgaaggaatggc

SEQ ID NO: 47 - DNA, 30 bp, Artificial Sequence
  Description of Artificial Sequence Synthetic primer
gaaatgaatgcagaaaaattagagcttgcg

SEQ ID NO: 48 - DNA, 30 bp, Artificial Sequence
  Description of Artificial Sequence Synthetic primer
tacaaatgcttgtttaccttcttctccacc

SEQ ID NO: 49 - DNA, 23 bp, Artificial Sequence
  Description of Artificial Sequence Synthetic primer
aattctcatcctacagcatctcc

SEQ ID NO: 50 - DNA, 20 bp, Artificial Sequence
  Description of Artificial Sequence Synthetic primer
tgaagaagcaccctctgttg

SEQ ID NO: 51 - DNA, 23 bp, Artificial Sequence
  Description of Artificial Sequence Synthetic primer
ggtgggtaaatgatgagtgtttc

SEQ ID NO: 52 - DNA, 25 bp, Artificial Sequence
  Description of Artificial Sequence Synthetic primer
tttttgatttgaccagcctttttag

SEQ ID NO: 53 - DNA, 20 bp, Artificial Sequence
  Description of Artificial Sequence Synthetic primer
tgtggaagaatccactgtcc

SEQ ID NO: 54 - DNA, 23 bp, Artificial Sequence
  Description of Artificial Sequence Synthetic primer
tctgtctctttcatttcctcaac

SEQ ID NO: 55 - DNA, 25 bp, Artificial Sequence
  Description of Artificial Sequence Synthetic primer
gaaggacgatacagacatttattgg

SEQ ID NO: 56 - DNA, 20 bp, Artificial Sequence
  Description of Artificial Sequence Synthetic primer
accgcaagttgaatagcaag

SEQ ID NO: 57 - DNA, 19 bp, Artificial Sequence
  Description of Artificial Sequence Synthetic primer
tcgcaagataaatgggaag

SEQ ID NO: 58 - DNA, 18 bp, Artificial Sequence
  Description of Artificial Sequence Synthetic primer
tcctcccgtaccaatcac

SEQ ID NO: 59 - DNA, 23 bp, Artificial Sequence
  Description of Artificial Sequence Synthetic primer
ttggtaaagtagaagcatcttgg

SEQ ID NO: 60 - DNA, 20 bp, Artificial Sequence
  Description of Artificial Sequence Synthetic primer
ccatcttatctgacccttgg

SEQ ID NO: 61 - DNA, 21 bp, Artificial Sequence
  Description of Artificial Sequence Synthetic primer
atatgtggttaggagccaatg

SEQ ID NO: 62 - DNA, 23 bp, Artificial Sequence
  Description of Artificial Sequence Synthetic primer
acaaatcgagtccaataatcacc

SEQ ID NO: 63 - DNA, 28 bp, Artificial Sequence
  Description of Artificial Sequence Synthetic primer
actactcgacaaggacgtttgtatttcc

SEQ ID NO: 64 - DNA, 20 bp, Artificial Sequence
  Description of Artificial Sequence Synthetic primer
ccatgtttcccatttgcttc

SEQ ID NO: 65 - DNA, 29 bp, Artificial Sequence
  Description of Artificial Sequence Synthetic primer
tgatatttatgaccaagattcaatatacg

SEQ ID NO: 66 - DNA, 22 bp, Artificial Sequence
  Description of Artificial Sequence Synthetic primer
gccatagctcaaggtcaatagg

SEQ ID NO: 67 - DNA, 20 bp, Artificial Sequence
  Description of Artificial Sequence Synthetic primer
ccaggtccatcctatgttgc

SEQ ID NO: 68 - DNA, 20 bp, Artificial Sequence
  Description of Artificial Sequence Synthetic primer
actacaatccgcgtgtctcc

SEQ ID NO: 69 - DNA, 22 bp, Artificial Sequence
  Description of Artificial Sequence Synthetic primer
cacgtgagtttacaccatacgc

SEQ ID NO: 70 - DNA, 21 bp, Artificial Sequence
  Description of Artificial Sequence Synthetic primer
ctagcacttgctctcatttcg

SEQ ID NO: 71 - DNA, 20 bp, Artificial Sequence
  Description of Artificial Sequence Synthetic primer
ggaacaactttcccacaagc

SEQ ID NO: 72 - DNA, 21 bp, Artificial Sequence
  Description of Artificial Sequence Synthetic primer
tcattcgtgaacccaataagc

SEQ ID NO: 73 - DNA, 20 bp, Artificial Sequence
  Description of Artificial Sequence Synthetic primer
aggaacgtgcgacgactagc

SEQ ID NO: 74 - DNA, 20 bp, Artificial Sequence
  Description of Artificial Sequence Synthetic primer
cacccgatatgttcaacagg

SEQ ID NO: 75 - DNA, 23 bp, Artificial Sequence
  Description of Artificial Sequence Synthetic primer
tttcttatccaacaggtgtatgc

SEQ ID NO: 76 - DNA, 21 bp, Artificial Sequence
  Description of Artificial Sequence Synthetic primer
gcgagtgtgacgttgataagc

SEQ ID NO: 77 - DNA, 20 bp, Artificial Sequence
  Description of Artificial Sequence Synthetic primer
tcaaacgttgccgaataagc

SEQ ID NO: 78 - DNA, 22 bp, Artificial Sequence
  Description of Artificial Sequence Synthetic primer
gacgattcgacacaacatatcg

SEQ ID NO: 79 - DNA, 21 bp, Artificial Sequence
  Description of Artificial Sequence Synthetic primer
ttggcattgtgttagatgagg

SEQ ID NO: 80 - DNA, 21 bp, Artificial Sequence
  Description of Artificial Sequence Synthetic primer
tgtgataaagccaaagcaacc

SEQ ID NO: 81 - DNA, 24 bp, Artificial Sequence
  Description of Artificial Sequence Synthetic primer
cagagcgtttcttaaatgaagagg

SEQ ID NO: 82 - DNA, 22 bp, Artificial Sequence
  Description of Artificial Sequence Synthetic primer
ttcatcatatgcctcaatttgg

SEQ ID NO: 83 - DNA, 20 bp, Artificial Sequence
  Description of Artificial Sequence Synthetic primer
aaaggcggtttagaatttgg

SEQ ID NO: 84 - DNA, 19 bp, Artificial Sequence
  Description of Artificial Sequence Synthetic primer
tgctgctctttacccatgc

SEQ ID NO: 85 - DNA, 20 bp, Artificial Sequence
  Description of Artificial Sequence Synthetic primer
tgtaaatgaacgccttgacc

SEQ ID NO: 86 - DNA, 21 bp, Artificial Sequence
  Description of Artificial Sequence Synthetic primer
ccgactccttctatcaattcc

SEQ ID NO: 87 - DNA, 23 bp, Artificial Sequence
  Description of Artificial Sequence Synthetic primer
aaacaatgttggatggtaaatgg

SEQ ID NO: 88 - DNA, 20 bp, Artificial Sequence
  Description of Artificial Sequence Synthetic primer
ccattcgcaaatttcactcc

SEQ ID NO: 89 - DNA, 20 bp, Artificial Sequence
  Description of Artificial Sequence Synthetic primer
ccgagctttaatggaaatgg

SEQ ID NO: 90 - DNA, 21 bp, Artificial Sequence
  Description of Artificial Sequence Synthetic primer
cctttaccttcagcctctagc

SEQ ID NO: 91 - DNA, 24 bp, Artificial Sequence
  Description of Artificial Sequence Synthetic primer
ggagaaacagatagtggtgaaagc

SEQ ID NO: 92 - DNA, 26 bp, Artificial Sequence
  Description of Artificial Sequence Synthetic primer
actctaaagccttgtattagatttcc

(19) United States
(12) Patent Application Publication
(10) Pub. No.: US 2006/0057603 A1
(43) Pub. Date: Mar. 16, 2006

(54) Detecting Bacillus anthracis

(75) Inventors: Lyndsay Radnedge, San Mateo, CA (US); Gary L. Andersen, Berkeley, CA (US)

Correspondence Address: Eddie E. Scott;Assistant Laboratory Counsel, Lawrence Livermore National Laboratory, P.O. Box 808, L-703, Livermore, CA, 94551, US

(73) Assignee: The Regents of the University of California

(21) Appl. No.: 11/075,059

(22) Filed: Mar. 7, 2005

Related U.S. Application Data
(60) Provisional application No. 60/552,224, filed on Mar. 11, 2004.

(51) Int. Cl. C12Q 1/68

(52) U.S. Cl. 435/6

(57) ABSTRACT

The present invention utilizes unique DNA signatures to identify Bacillus anthracis. The DNA signatures can also be used in other types of detection. These signatures have been found to be unique to Bacillus anthracis, and can be used to understand how these DNA regions are being used to make this species different from closely related Bacillus species. Mechanisms of virulence, invasion, evolution, interactions with other microbes, antibiotic resistance etc. can be investigated with these regions once they are known. The method comprises using a multiplex polymerase chain reaction analysis of separate loci on the Bacillus anthracis chromosome to produce a set of chromosomal DNA signatures, and using at least one of the chromosomal DNA signatures to detect genetically the Bacillus anthracis.

### CROSS-REFERENCE TO RELATED APPLICATIONS

This application claims the benefit of U.S. Provisional Patent Application No. 60/552,224 filed Mar. 11, 2004 and titled “DNA Diagnostics for Bacillus Anthracis.” U.S. Provisional Patent Application No. 60/552,224 filed Mar. 11, 2004, titled “DNA Diagnostics for Bacillus Anthracis” is incorporated herein by this reference.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to DNA diagnostics and more particularly to detecting Bacillus anthracis.

2. State of Technology

U.S. Pat. No. 6,664,104 to Farzad Pourahmadi et al and assigned to Cepheid for a “Device Incorporating a Microfluidic Chip for Separating Analyte from a Sample” issued Dec. 16, 2003 provides the following state of technology information, “Abstract: A device for separating an analyte from a fluid sample comprises a cartridge incorporating a flow-through microfluidic chip. The microfluidic chip includes an extraction chamber having an array of microstructures for capturing the analyte and for subsequently releasing the captured analyte into an elution fluid. Each of the microstructures has an aspect ratio of at least 2:1. The cartridge also includes channels and at least one low controller (e.g., one or more valves) for directing the flow of the sample and elution fluid through the microfluidic chip. The cartridge may optionally include a lysing region for lysing sample components (e.g., cells spores, or microorganisms), a waste chamber for storing waste fluid, and reaction or detection chambers for amplifying or detecting the analyte.”

U.S. Patent Application No. 2002/0045246 to William A. McMillan et al and assigned to Cepheid for a “Device for Lysing Cells, Spores, or Microorganisms” published Apr. 18, 2002 provides the following state of technology information, “Abstract: A device for use with an ultrasonic transducer to lyse components of a fluid sample comprises a cartridge having a lysing chamber, an inlet port in fluid communication with the lysing chamber, and an outlet port for exit of the sample from the lysing chamber. The inlet and outlet ports are positioned to permit flow of the sample through the lysing chamber, and the chamber contains at least one solid phase for capturing the sample components to be lysed as the sample flows through the chamber. The lysing chamber is defined by at least one wall having an external surface for contacting the transducer to effect the transfer of ultrasonic energy to the chamber.”

U.S. Pat. No. 6,569,630 to Jeevalatha Vivekananda and Johnathan L. Kiel for “Methods and Compositions for Aptamers Against Anthrax” issued May 27, 2003 provides the following state of technology information, “There is a great need for the development of methods, compositions and apparatus capable of detecting and identifying known or unknown chemical and biological agents (herein referred to as analytes), which include but are not limited to nucleic acids, proteins, illicit drugs, explosives, toxins, pharmaceuticals, carcinogens, poisons, allergens, contaminants, pathogens and infectious agents . . . . Within the field of biological warfare, there is a great need for a rapid, sensitive method to detect and identify pathogenic spores of Bacillus anthrax (hereafter “anthrax”). Anthrax is a highly pathogenic biological agent that is relatively simple to produce and distribute in the field. Present methods for detection of anthrax are not sufficiently rapid, sensitive, and robust to allow early detection of exposure to anthrax under field conditions, such as might be encountered on a battlefield. No good method presently exists for neutralization of anthrax under field conditions.”

U.S. Patent Application No. 2003/0124556 by David, J. Ecker for “Method for Rapid Detection and Identification of Bioagents” published Jul. 3, 2003 provides the following state of technology information, “Rapid and definitive microbial identification is desirable for a variety of industrial, medical, environmental, quality, and research reasons. Traditionally, the microbiology laboratory has functioned to identify the etiologic agents of infectious diseases through direct examination and culture of specimens. Since the mid-1980s, researchers have repeatedly demonstrated the practical utility of molecular biology techniques, many of which form the basis of clinical diagnostic assays. Some of these techniques include nucleic acid hybridization analysis, restriction enzyme analysis, genetic sequence analysis, and separation and purification of nucleic acids (See, e.g., J. Sambrook, E. F. Fritsch, and T. Maniatis, Molecular Cloning: A Laboratory Manual, 2nd Ed., Cold Spring Harbor Laboratory Press, Cold Spring Harbor, N.Y., 1989). These procedures, in general, are time-consuming and tedious. Another option is the polymerase chain reaction (PCR) or other amplification procedure which amplifies a specific target DNA sequence based on the flanking primers used. Finally, detection and data analysis convert the hybridization event into an analytical result.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention utilizes unique DNA signatures to identify Bacillus anthracis. The DNA signatures can also be used in other types of detection. These signatures have been found to be unique to Bacillus anthracis, and the signatures can be used to help understand how these DNA regions are being used to make this species different from closely related Bacillus species. Mechanisms of virulence, invasion, evolution, interactions with other microbes, antibiotic resistance etc. can be investigated with these regions. These DNA signatures can also be used to identify unique regions that could be targets for protein-based diagnostics and research; and to identify targets for vaccine development, protein recognition mechanisms, basic research to understand evolutionary aspects of proteins, and how they are used among different applications. In one embodiment, the present invention provides a method of detecting Bacillus anthracis, comprising selecting at least one individual locus on a Bacillus anthracis chromosome, using a multiplex polymerase chain reaction analysis of the individual loci on a Bacillus anthracis chromosome to produce at least one chromosomal DNA signature, and using the at least one chromosomal DNA signature to detect genetically the Bacillus anthracis.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 shows a Phylogenetic tree of B. anthracis, B. cereus, and B. thuringiensis isolates.

FIG. 2 is a graphic representation of the locations of the tester-specific clones on the B. anthracis A2012 genome.

FIG. 3 shows a multiplex analysis of four separate loci on the B. anthracis genome. Four B. anthracis-specific primers yielded predicted products of 133, 163, 196, and 241 bp.

### DETAILED DESCRIPTION OF THE INVENTION

Referring now to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

The present invention provides a method of detecting Bacillus anthracis, comprising selecting at least one individual locus on a Bacillus anthracis chromosome, using a multiplex polymerase chain reaction analysis of the individual locus on a Bacillus anthracis chromosome to produce at least one chromosomal DNA signature, and using at least one chromosomal DNA signature to detect genetically the Bacillus anthracis. The article, “Genome Differences That Distinguish Bacillus anthracis from Bacillus cereus and Bacillus thuringiensis” by Lyndsay Radnedge, Peter G. Agron, Karen K. Hill, Paul J. Jackson, Lawrence O. Ticknor, Paul Keim, and Gary L. Andersen, in Applied and Environmental Microbiology, May 2003, Volume 69, Issue 5, provides information about the present invention. The article, “Genome Differences That Distinguish Bacillus anthracis from Bacillus cereus and Bacillus thuringiensis” is incorporated herein in its entirety by this reference.

The three species of the group 1 bacilli, Bacillus anthracis, B. cereus, and B. thuringiensis, are genetically very closely related. All inhabit soil habitats but exhibit different phenotypes. B. anthracis is the causative agent of anthrax and is phylogenetically monomorphic, while B. cereus and B. thuringiensis are genetically more diverse. An amplified fragment length polymorphism analysis demonstrates genetic diversity among a collection of non-anthrax-causing Bacillus species, some of which show significant similarity to B. anthracis. Suppression subtractive hybridization was then used to characterize the genomic differences that distinguish three of the non-anthrax-causing bacilli from B. anthracis Ames. Ninety-three DNA sequences that were present in B. anthracis but absent from the non-anthrax-causing Bacillus genomes were isolated. Furthermore, 28 of these sequences were not found in a collection of 10 non-anthrax-causing Bacillus species but were present in all members of a representative collection of B. anthracis strains. These sequences map to distinct loci on the B. anthracis genome and can be assayed simultaneously in multiplex PCR assays for rapid and highly specific DNA-based detection of B. anthracis.

In one embodiment, the present invention provides a method that comprises using a multiplex polymerase chain reaction analysis of four separate loci on the Bacillus anthracis chromosome to produce a set of twenty eight chromosomal DNA signatures, and using at least one of the twenty eight chromosomal DNA signatures to detect genetically the Bacillus anthracis. The present invention utilizes unique DNA signatures to identify Bacillus anthracis. Systems for utilizing DNA signatures for identification are known in the art. For example, systems for utilizing DNA signatures for identification are described in U.S. Patent Application No. 2003/0124556 and the other publications identified in the patent application. U.S. Patent Application No. 2003/0124556 and the other publications are incorporated herein by this reference. The DNA signatures can also be used in other types of detection. These signatures have been found to be unique to Bacillus anthracis, and can be used to understand how these DNA regions are being used to make this species different from closely related Bacillus species. Mechanisms of virulence, invasion, evolution, interactions with other microbes, antibiotic resistance etc. can be investigated with these regions once they are known. These DNA signatures can also be used to identify unique regions that could be targets for protein-based diagnostics and research and to identify targets for vaccine development, protein recognition mechanisms, basic research to understand evolutionary aspects of proteins and how they are used among different applications. The DNA signatures fulfill the national need better, rapid DNA diagnostics that are highly specific for Bacillus anthracis.

Bacillus anthracis, B. cereus, and B. thuringiensis are genetically very closely related members of the group 1 bacilli, a fact which has led to the proposal that they should be considered a single species. B. cereus is frequently isolated as a contaminant of various foods and can occasionally be an opportunistic human pathogen. B. thuringiensis has been widely exploited in agriculture as an insecticide by virtue of the presence of plasmid-borne crystal toxin genes. B. anthracis is a virulent pathogen of mammals and is the causative agent of anthrax. All three species are readily isolated from soil environments.

Extensive genetic diversity among environmental isolates of B. cereus and B. thuringiensis has been demonstrated by pulsed-field gel electrophoresis, multienzyme electrophoresis, and amplified fragment length polymorphism (AFLP) analysis. AFLP analysis proved sensitive enough to classify B. cereus and B. thuringiensis into five phylogenetic groups. Significantly, the American Type Culture Collection (ATCC) reference strains of B. cereus and B. thuringiensis did not seem to be represented in the collection of environmental isolates used in that study. There was little correlation between species designation and the five phylogenetic groups identified by AFLP analysis, indicating significant genetic variability within and between B. cereus and B. thuringiensis. Furthermore, in two separate studies, the B. anthracis strains appeared to cluster together with a group containing periodontal B. cereus pathogens.

In contrast to the genomic diversity within B. cereus and B. thuringiensis, B. anthracis appears to be genetically clonal. Genetic identity among different B. anthracis isolates has necessitated fastidious analysis of their genomes for strain identification. Accurate discrimination is now possible by analysis of variable-number tandem repeats, which enumerates small tandem repeats at several locations in the B. anthracis genome. A collection of 426 B. anthracis environmental isolates that contains representatives from worldwide origins was subdivided by variable-number tandem repeat analysis into just six genetically distinct groups.

Several strains of B. cereus and B. thuringiensis which appear to be genetically related to B. anthracis have been isolated. Isolation and characterization of the genome regions unique to B. anthracis will provide clues to its genetic relationship to these strains and ultimately direct pathogenicity studies. AFLP analysis provides a rapid method for measuring phylogenetic distances. AFLP analysis generates a strain-specific fingerprint of amplified DNA fragments that demonstrate genomic variations in a microbial population based on an analysis of a portion of their genome sequences and DNA fragment length polymorphisms. The great advantage of AFLP analysis is its ability to analyze rapidly many loci, resulting in a phylogenetic resolution higher than those obtained with other methods. However, AFLP analysis provides little information about the genetic differences responsible for these polymorphisms. Suppression subtractive hybridization (SSH) is a highly efficient technique for the isolation and characterization of the large genomic differences that often drive bacterial genome evolution. SSH reveals DNA sequence differences that are responsible for many AFLPs.

Unique genomic differences can be exploited as “DNA signatures” for the discrimination of B. anthracis from its closest relatives. Plasmid-encoded toxin genes have proved a useful source of targets for rapid DNA-based assays. However, a plasmid-based test may not detect the occurrence of non-plasmid-containing strains of B. anthracis, which have been isolated from the environment. Furthermore, plasmids can be readily engineered and can be transferred to other bacteria for heterologous gene expression, and there is concern that pXO1 and pXO2 sequences that are present in other Bacillus strains may be encountered in previously uncharacterized genomes. There are many published examples of chromosomal regions that can be examined for B. anthracis identification, but all require time-consuming downstream analysis. Two recent examples of real-time PCR targeting of the rpoB gene of B. anthracis were useful but reported examples of false-positive results with some strains of B. cereus and targeted only a single locus.

This Application demonstrates the phylogenetic relationships of a collection of non-anthrax-causing Bacillus species to each other and to B. anthracis, as determined by AFLP analysis. SSH with B. anthracis and three of these close relatives identified a set of unique DNA regions that represent genomic differences between B. anthracis and these non-anthrax-causing Bacillus species. One immediate product of these experiments was a robust set of chromosomal DNA signatures which are capable of quickly detecting all six genetically distinct groups of B. anthracis and which can be used with any rapid DNA-based detection platform. A multiplex PCR analysis with four separate loci of the B. anthracis chromosome provides a rapid and highly specific means for the identification of B. anthracis.

Materials and Methods—AFLP analysis of DNA samples. AFLP analysis was accomplished as previously described. Briefly, DNA (100 ng) was digested with EcoRI and MseI, and the resulting fragments were ligated to double-stranded adaptors. The digested and ligated DNA was then amplified by PCR with EcoRI and MseI +0/+0 primers. The +0/+0 PCR product was analyzed by agarose gel electrophoresis to determine the size range of amplified fragments. Three microliters was used in subsequent selective amplifications with 6-carboxyfluorescein-labeled +1/+1 primers EcoRI-C (5′-GTAGACTGCGTACCAATTCC-3′, SEQ ID NO: 29) and MseI-G (5′-GACGATGAGTCCTGAGTAAG-3′, SEQ ID NO: 30). Selective amplifications were performed with 20-μl reaction mixtures. The resulting products (0.5 to 1.0 μl) were mixed with a solution containing the DNA size standards Genescan-500 (Applied Biosystems, Inc., Foster City, Calif.) and MapMarker-400 (BioVentures, Inc., Murfreesburo, Tenn.), both labeled with N,N,N,N-tetramethyl-6-carboxyrhodamine. Following heat denaturation at 90° C. for 2 min, the reaction mixtures were loaded into a 5% Long Ranger DNA sequencing gel (BioWhittaker Molecular Applications, Rockland, Me.) and visualized with an ABI 377 automated fluorescence sequencer (Applied Biosystems). GeneScan analysis software (Applied Biosystems) was used to determine the lengths of the sample fragments by comparison to the DNA fragment length size standards included with each sample. AFLP data analysis was performed as described by Ticknor et al.

Preparation of genomic DNA—The bacterial strains used for genomic DNA preparation and their geographic origins, where known, are listed in Table 1. DNA was extracted from cell pellets from overnight cultures grown in L broth by using a MasterPure DNA purification kit (Epicentre, Madison, Wis.) according to the manufacturer's instructions. Genomic DNA was prepared from enteric bacteria by using Wizard Genomic DNA Preps (Promega, Madison, Wis.) according to the manufacturer's instructions. Human genomic DNA was purchased from Clontech (Palo Alto, Calif.), and bovine genomic DNA was purchased from Novagen (Madison, Wis.). Soil DNA was extracted by using an UltraClean soil DNA kit (MoBio, Solana Beach, Calif.) according to the manufacturer's instructions. DNA isolated from organisms present in the air was prepared from filters as previously described. Both soil and air filter DNAs were prepared from samples originating in Livermore, Calif. After ethanol precipitation, all genomic DNAs were dissolved in 10 mM Tris HCl (pH 8.0) to a concentration of approximately 0.2 μg/ml.

TABLE 1
Strains used in this Study

| | Phylogenetic | Geographic |
|---|---|---|
| Strain | Group | Origin(s) |
| B. anthracis | | |
| G3 | A1.a | North America, Europe, |
| | | Brazil |
| G20 | A1.a | Italy |
| G25 | A1.b | U.S. |
| G29 | A2 | Pakistan |
| G38 | A3.a | Germany |
| G62 (Ames) | A3.b | U.S., U.K. |
| G67 | A3 | South Africa |
| G77 (Vollum) | A4 | U.K., Spain, Zimbabwe |
| G80 | B2 | France |
| G87 | B1 | South Africa |
| Non-anthrax-causing | | |
| Bacillus species | | |
| B. cereus ATCC 14579 | NA | NR |
| B. cereus ATCC 4342 | NA | NR |
| B. cereus D17 | NA | U.S. |
| B. cereus 3A | NA | U.S. |
| B. cereus S2-8 | NA | U.S. |
| B. cereus F1-15 | NA | U.S. |
| B. thuringiensis 97-27 | NA | Bosnia |
| B. thuringiensis Al Hakam | NA | Iraq |
| B. thuringiensis ATCC 10792 | NA | Israel |
| B. thuringiensis HD-571 | NA | USDA |

SSH—Genome comparisons by SSH were performed as previously described, except for the differences noted below. Briefly, the tester-specific DNA was digested with restriction endonucleases (RsaI and MseI; New England Biolabs, Beverly, Mass.) used according to the manufacturer's instructions. The fragments were first marked by ligation to specialized oligonucleotide adaptors. When the marked DNA was denatured and hybridized to excess unmarked driver-specific DNA that had been digested with the same enzymes, most tester-specific sequences formed heterohybrids with the driver. Some tester-specific sequences, however, self-hybridized to form amplifiable fragments that were then enriched by PCR, cloned, and sequenced. Agarose gel electrophoresis determined that the restriction endonucleases MseI and RsaI cut the genomic DNA to generate fragments in the optimal size range (200 to 1,000 bp). Modified adaptors were constructed to allow for subtractions with MseI-digested DNA as previously described. Namely, adaptor 1 was formed by annealing the “adaptor 1 long” oligonucleotide with the oligonucleotide 5′-TAACCTGCCCGG (SEQ ID NO: 31) to form an adaptor with appropriate cohesive ends. Adaptor 2 was formed by annealing the “adaptor 2 long” oligonucleotide with the oligonucleotide 5′-TAACCTCGGCCG (SEQ ID NO: 32). T4 DNA ligase (New England Biolabs) was inactivated by incubation at 72° C. for 20 min. Three separate SSH experiments were performed with B. anthracis Ames as the tester and three of the non-anthrax-causing Bacillus strains listed in Table 1 as the drivers: B. cereus ATCC 14579, B. cereus 3A, and B. thuringiensis Al Hakam.

DNA sequencing—Nonpurified PCR products were cloned by using a pGEM-T Easy TA cloning kit (Promega). Recombinant clones were picked by using a BioPick automated colony picker (BioRobotics, Woburn, Mass.), and plasmid templates were prepared by boiling lysis and magnetic bead capture with a high-throughput procedure. Sequencing of plasmid templates was performed by using an Applied Biosystems Big-Dye Terminator system and either an ABI 377 or an ABI 3700 automated sequencer. The sequencing primers used were 5′-TGTAAAACGACGGCCAGT (forward) (SEQ ID NO: 33) and 5′-CAGGAAACAGCTATGACC (reverse) (SEQ ID NO: 34). The resulting data were analyzed by using ABI sequencing analysis software, version 3.2, and then assembled and edited by using Phred, Phrap, and Consed 7.0. BLAST searches with the tester-specific DNA sequences were performed by using the National Center for Biotechnology Information website www.ncbi.nlm.nih.gov/BLAST identity was considered significant only when the expected probability of a fortuitous match (E value) was less than 10⁻⁴. Comparison of the sequence candidates to plasmids pXO1 and pXO2 and determination of their coordinates on the published B. anthracis genome were performed by using the crossmatch program, which is part of the Consed software package.

PCRs.—Oligonucleotide primers were designed from the putative tester-specific sequences and were supplied by Sigma-Genosys (The Woodlands, Tex.) or Invitrogen (Carlsbad, Calif.). The primers were designed by using Primer3 software; they had a melting temperature of >65° C., contained no more than three identical consecutive nucleotides, and possessed a two-nucleotide 3′ GC clamp. The primers were initially screened against genomic DNAs from both the tester and the driver. To determine whether a primer pair was tester specific, 1 ng each of tester- and driver-specific DNAs was used as a template in PCRs with Accuprime polymerase, primers at 10 μM (Invitrogen), and the following cycling parameters: 94° C. for 15 s, 65° C. for 15 s, and 72° C. for 30 s for 32 cycles. The products were visualized on a 1.5% agarose gel run in 0.5× Tris-borate-EDTA; if a product was present with tester-specific DNA as a template and absent with driver-specific DNA, then that sequence was designated tester specific. The tester-specific oligonucleotides were then used to prime PCRs with the B. anthracis, B. cereus, and B. thuringiensis strains listed in Table 1 and the same reaction conditions as those described above. The integrity of the genomic DNA template was tested in all PCRs with primers specific for a region of the 23S gene conserved in Bacillus species: 5′-CTACCTTAGGACCGTTATAGTTAC (SEQ ID NO: 35) and 5′-AGGTAGGCGAGGAGAGAATCC (SEQ ID NO: 36). Multiplex PCRs were performed as described above, except that template DNA was added to a final concentration of 10 ng/μl. The primers (see Table 5) were used at the following concentrations: 23S primers at 3 μM (288 bp), dhp73.017 (241 bp) at 20 μM, dhp73.019 (196 bp) at 10 μM, dhp61.183 (163 bp) at 5 μM, and dhp77.002 (133 bp) at 20 μM.

TABLE 5

Primers designed from the twenty-eight B. anthracis specific DNA signatures, and
their coordinates on the B. anthracis A2012 genome (Accession number: NC_003995).

| | | | | | | |
|---|---|---|---|---|---|---|
| | Length | | | | Pre- | |
| | of | | | | dicted | |
| | Differ- | | | | PCR | |
| | ence | GC | Reverse | Reverse | pro- | |
| Fragment | Product | Per- | Primer | Primer | duct | |
| Name | (bp) | cent | sequence | sequence | (bp) | Strand⁽¹⁾ |

Tester:B anthracis Ames; Driver: B. centus ATCC14579

| | | | | | | | |
|---|---|---|---|---|---|---|---|
| 1 | R.Ctg132 | 94 | 36.7 | ACCTTTTGCTAAAGTGATTTCACCTTTTG | ACTGAAGTTGTTTCACCAGAGGGTATTGA | 90 | C |
| 2 | R.Ctg139 | 268 | 35.6 | CAAAGTGGCAAAAGGGGAATTTAGA | CGCCCAACAACTTGAGAGCCTACAA | 157 | C |
| 3 | R.Ctg152 | 425 | 32.8 | TCCCAATACATATGAGCGATTCGCC | GGTGGTATGTAAAAATCAAATAGGACGGA | 155 | |
| 4 | R.Ctg177 | 612 | 31.9 | TTGGATCAGCGTTTCTGAATTCAGC | TCCCCATATCGCTCAATTCCATCTA | 160 | |
| 5 | M.Ctg032 | 342 | 34.7 | GGCCCCATAACTTGATTTCTCTTCA | ATCTTGCTCCAGCTGAAGGAATGGC | 151 | |
| 6 | M.Ctg037 | 151 | 36.2 | GAAATGAATGCAGAAAAATTAGAGCTTG | TACAAATGCTTGTTTACCTTCTTCTCCAC | 135 | |
| | | | | CGC | | | |

Tester B. anthracis Ames; Driver; Bacillus Al Hakam

| | | | | | | | |
|---|---|---|---|---|---|---|---|
| 7 | dhp61.159 | 420 | 31.7 | AATTCTCATCCTACAGCATCTCC | TGAAGAAGCACCCTCTGTTG | 151 | |
| 8 | dhp61.178 | 479 | 31.3 | GGTGGGTAAATGATGAGTGTTTC | TTTTTGATTTGACCAGCCTTTTTAG | 145 | C |
| 9 | dhp61.161 | 1597 | 36.8 | TGTGGAAGAATCCACTGTCC | TCTGTCTCTTTCATTTCCTCAAC | 110 | C |
| 10 | dhp61.183² | 645 | 31.6 | GAAGGACGATACAGACATTTATTGG | ACCGCAAGTTGAATAGCAAG | 163 | C |
| 11 | dhp64.177 | 482 | 43.6 | TCGCAAGATAAATGGGAAG | TCCTCCCGTACCAATCAC | 101 | C |
| 12 | dhp64.188 | 531 | 35.8 | TTGGTAAAGTAGAAGCATCTTGG | CCATCTTATCTGACCCTTGG | 134 | C |
| 13 | dhp64.252 | 451 | 36.6 | ATATGTGGTTAGGAGCCAATG | ACAAATCGAGTCCAATAATCACC | 137 | |
| 14 | dhp64.208 | 596 | 42.0 | ACTACTCGACAAGGACGTTTGTATTTCC | CCATGTTTCCCATTTGCTTC | 152 | C |

Tester: B.anthracis Ames; Driver: B. census 3A

| | | | | | | | |
|---|---|---|---|---|---|---|---|
| 15 | dhp77.002 | 248 | 33.9 | TGATATTTATGACCAAGATTCAATATACG | GCCATAGCTCAAGGTCAATAGG | 133 | C |
| 16 | dhp77.003 | 229 | 41.6 | CCAGGTCCATCCTATGTTGC | ACTACAATCCGCGTGTCTCC | 178 | |
| 17 | dhp77.004 | 300 | 38.1 | CACGTGAGTTTACACCATACGC | CTAGCACTTGCTCTCATTTCG | 243 | |
| 18 | dhp77.047 | 175 | 42.4 | GGAACAACTTTCCCACAAGC | TCATTCGTGAACCCAATAAGC | 148 | C |
| 19 | dhp77.056 | 179 | 42.0 | AGGAACGTGCGACGACTAGC | CACCCGATATGTTCAACAGG | 167 | C |
| 20 | dhp73.001 | 490 | 32.2 | TTTCTTATCCAACAGGTGTATGC | GCGAGTGTGACGTTGATAAGC | 211 | C |
| 21 | dhp73.009 | 438 | 30.6 | TCAAACGTTGCCGAATAAGC | GACGATTCGACACAACATATCG | 166 | C |
| 22 | dhp73.011 | 387 | 33.3 | TTGGCATTGTGTTAGATGAGG | TGTGATAAAGCCAAAGCAACC | 204 | |
| 23 | dhp73.016 | 467 | 28.9 | CAGAGCGTTTCTTAAATGAAGAGG | TTCATCATATGCCTCAATTTGG | 166 | C |
| 24 | dhp73.017 | 497 | 33.8 | AAAGGCGGTTTAGAATTTGG | TGCTGCTCTTTACCCATGC | 241 | C |
| 25 | dhp73.019 | 327 | 38.5 | TGTAAATGAACGCCTTGACC | CCGACTCCTTCTATCAATTCC | 196 | |
| 26 | dhp73.022 | 244 | 39.8 | AAACAATGTTGGATGGTAAATGG | CCATTCGCAAATTCACTCC | 144 | C |
| 27 | dhp73.026 | 548 | 27.7 | CCGAGCTTTAATGGAAATGG | CCTTTACCTTCAGCCTCTAGC | 236 | |
| 28 | dhp73.029 | 242 | 30.6 | GGAGAAACAGATAGTGGTGAAAGC | ACTCTAAAGCCTTGTATTAGATTTCC | 133 | |

| | |
|---|---|
| BLASTX, against B. anthracis A2012 genome (NC_003995) | BLASTX against GenBank database |

| | | | | | | | |
|---|---|---|---|---|---|---|---|
| 5′ Co- | 3′ Co- | BA A2012 | BA A2012 | | | Accession | |
| ordinate | ordinate | BLASTX | Gene | E Value | Description | Number | E Value |

Tester:B anthracis Ames; Driver: B. centus ATCC14579

| | | | | | | | | |
|---|---|---|---|---|---|---|---|---|
| 1 | 1,396,681 | 1,396,592 | NP_654829 | BA_489 | 3.0E−09 | S-layer protein | P49051 | 3.0E−09 |
| | | | | | | [Bacillus anthracis | | |
| 2 | 3,923,298 | 3,923,035 | NP_657605 | BA_4245 | 6.0E−44 | Hypothetical phage protein | NP_606634 | 9.0E−05 |
| | | | | | | (Streptococcus pyogenes) | | |
| 3 | 4,227,290 | 4,227,710 | NP_657938 | BA_4578 | 4.0E−12 | Novel | | |
| 4 | 996,045 | 996,652 | NP_654415 | BA_1055 | 4.0E−89 | Novel | | |
| 5 | 344,770 | 345,109 | NP_653725 | BA_0365 | 2.0E−63 | Galactosyltranserase | NP_349903 | 7.0E−08 |
| | | | | | | [Clostridium acetobutylicum] | | |
| 6 | 690,508 | 990,656 | NP_654413 | BA_1053 | 1.0E−08 | Novel | | |

Tester B. anthracis Ames; Driver; Bacillus Al Hakam

| | | | | | | | | |
|---|---|---|---|---|---|---|---|---|
| 7 | 4,870,037 | 4,870,457 | NP_658848 | BA_5288 | 1.0E−29 | Novel | — | — |
| 8 | 349,630 | 349,192 | NP_653729 | BA_0369 | 1.0E−76 | Novel | — | — |
| 9 | 4,209,359 | 4,207,762 | NP_657910 | BA_4550 | 1.0E−179 | BH0961: unknown conserved | NP_241827 | 4.0E−04 |
| | | | | | | protein (Bacillus | | |
| | | | | | | halodurans) | | |
| 10 | 186,609 | 185,965 | NP_653563 | BA_0203 | 4.0E−42 | Novel | — | — |
| 11 | 3,427,212 | 3,426,731 | NP_657087 | BA_3727 | 8.0E−82 | Penicillin binding protein | BAB69972 | 1.0E−67 |
| | | | | | | [Bacillus cerus] | | |
| 12 | 4,203,832 | 4,203,302 | NP_657908 | BA_4548 | 1.0E−100 | Novel | — | — |
| 13 | 192,470 | 192,922 | NP_653575 | BA_0215 | 9.0E−47 | Putative phage laminase | NP_562034 | 5.0E−08 |
| | | | | | | [Clostridium perfringens] | | |
| 14 | 3,951,710 | 3,951,115 | NP_657641 | BA_4281 | 7.0E−83 | Cytosine-specific methyl- | NP_244375 | 2.0E−03 |
| | | | | | | transferase [Bacillus | | |
| | | | | | | halodurans] | | |

Tester: B.anthracis Ames; Driver: B. census 3A

| | | | | | | | | |
|---|---|---|---|---|---|---|---|---|
| 15 | 980,947 | 961,187 | NP_654400 | BA_1040 | 5.0E−42 | Phage terminese Phi SLT | NP_075501 | 1.0E−06 |
| | | | | | | [Staphylococcus aureus] | | |
| 16 | 3,701,825 | 3,702,023 | Intergenic | — | — | Novel | — | — |
| 17 | 1,292,142 | 1,292,441 | NP_654715 | BA_1355 | 7.0E−52 | Novel | — | — |
| 18 | 965,870 | 965,695 | NP_654367 | BA_1007 | 1.0E−26 | hypothetical protein nanH | 140868 | 2.0E−06 |
| | | | | | | region [Clostridium | | |
| | | | | | | perfringens] | | |
| 19 | 3,927,774 | 3,927,596 | Intergenic | — | — | Novel | — | — |
| 20 | 699,664 | 699,175 | NP_654100 | BA_0740 | 2.0E−15 | L-glutamine-D-fructose-6- | BAA04741 | 2.1E−12 |
| | | | | | | phosphate amidotransferase | | |
| | | | | | | [Bacillus subt. | | |
| 21 | 3,223,428 | 3,222,991 | NP_656951 | BA_3491 | 3.0E−22 | Novel | — | — |
| 22 | 2,472,346 | 2,472,732 | NP_655986 | BA_2526 | 1.0E−67 | ABC transporter (ATP- | NP_561360 | 5.0E−32 |
| | | | | | | binding protein) | | |
| | | | | | | [Clostridium perfringens] | | |
| 23 | 3,945,582 | 3,945,125 | NP_657634 | BA_4274 | 8.0E−72 | Novel | — | — |
| 24 | 4,198,535 | 4,198,039 | NP_657898 | BA_4538 | 1.0E−82 | Novel | — | — |
| 25 | 3,920,569 | 3,920,882 | NP_657604 | BA_4244 | 8.0E−45 | Novel | — | — |
| 26 | 4,205,247 | 4,205,004 | NP_657908 | BA_4548 | 1.0E−42 | Novel | — | — |
| 27 | 1,472,742 | 1,473,289 | Intergenic | — | — | BH2522: unknown conserved | NP_243388 | 1.0E−13 |
| | | | | | | protein [Bacillus | | |
| | | | | | | halodurans] | | |
| 28 | — | — | — | — | — | Novel | — | — |

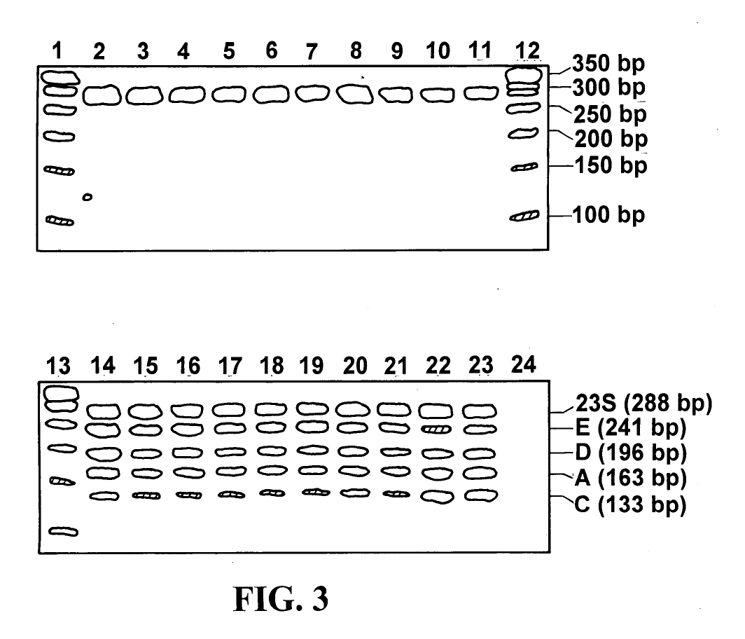

⁽¹⁾C Indicates identity to the complimentary strand.
⁽²⁾The underlined primers indicate those used in the multiplex PCR assays shown in FIG. 3.

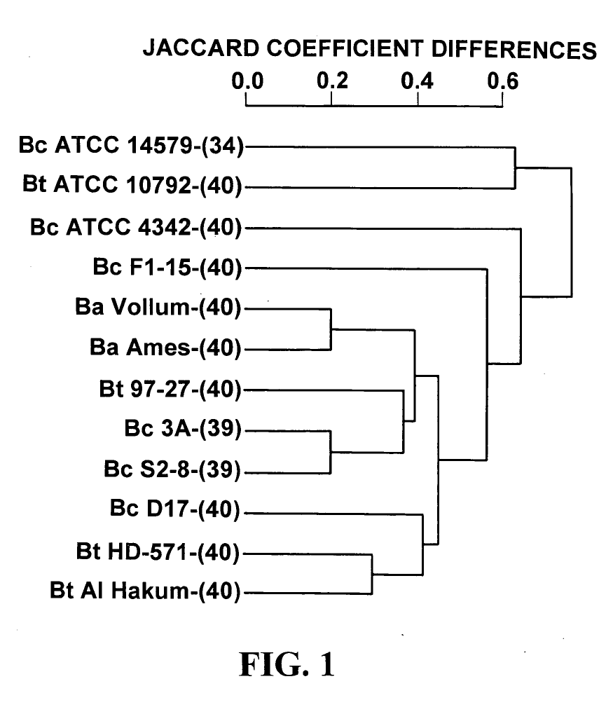

Results—Genetic relatedness of B. anthracis to B. cereus and B. thuringiensis, as determined by AFLP.—The phylogenetic tree derived from the fluorescent AFLP data is shown in FIG. 1.

Referring now to FIG. 1, a Phylogenetic tree of B. anthracis, B. cereus, and B. thuringiensis isolates is shown. The phylogenetic tree was derived from fluorescent AFLP analyses of 12 B. anthracis (Ba), B. cereus (Bc), and B. thuringiensis (Bt) isolates. The phylogenetic tree was based on 34 to 40 amplified DNA fragments per sample (the number per sample is shown in parentheses) generated from EcoRI/MseI digestion of genomic DNAs. Jaccard coefficients for the fragments common in three replicate gels for each isolate were analyzed by using the unweighted pair-group method with arithmetic means to produce the dendrogram.

The Bacillus isolates used in this study were chosen from a previous AFLP study of over 300 Bacillus isolates (K. K. Hill and P. J. Jackson, unpublished data). They included several B. cereus and B. thuringiensis isolates that were found to be the most closely related to B. anthracis and two more distantly related B. cereus and B. thuringiensis strains (ATCC type strains 14579 and 10792, respectively). The other Bacillus isolates represented on the tree include pathogenic and nonpathogenic isolates; details for these isolates are shown in Table 1. B. anthracis is represented in this tree by the Ames and Vollum strains. A large number of B. anthracis isolates were previously tested and found to possess AFLP profiles indistinguishable from those of these two strains (data not shown). The isolate most closely related to B. anthracis is B. thuringiensis 97-27. This isolate was collected from the wound of a French soldier and was shown to be capable of infecting and killing immunocompetent mice in subsequent studies. The three other B. thuringiensis isolates on the tree are HD-571, obtained from the U.S. Department of Agriculture B. thuringiensis collection; Al Hakam, collected by the United Nations Special Commission at a suspected bioweapons facility in Iraq; and the ATCC type strain (ATCC 10792). The B. cereus isolates also include pathogenic and nonpathogenic members of this species. Three B. cereus isolates (F1-15, 3A, and D17) were collected from food sources that caused human illness, and one (S2-8) was a soil isolate. The B. cereus ATCC type strain (ATCC 14579) and ATCC 4342, an isolate from milk, were also included.

Data were analyzed by using three or more replicates of the AFLP profiles to compare each isolate to the other isolates. AFLP fragments that appeared in every replicate for an isolate were used to define a fingerprint for that isolate. Each gel contained one or more control DNA samples to allow comparisons of profiles on the different gels. Jaccard coefficients for the fingerprints of the isolates being compared were analyzed by using an unweighted pair-group method with arithmetic means to produce the dendrogram. Based on an analysis of control DNA samples, the variability within the analysis was less than about 0.2 for the Jaccard coefficients. Any differences below this level therefore may be due to experimental or analytical variability and cannot be considered significant. Differences above this level are reproducible and carry a high degree of confidence.

Isolation of tester-specific DNA sequences by SSH.—SSH identifies DNA sequences that are specific to one genome (tester) and absent from the other genome (driver). Typically these DNA sequences range in length from 200 to 1,000 bp and are referred to as difference products. Three subtractions were undertaken to increase the yield and representation of B. anthracis-specific regions. B. cereus ATCC 14579 was selected because it represents one of the two most distantly related strains in this study and would be most likely to yield difference products. B. cereus 3A was chosen because it represents one of the closest relatives of B. anthracis, while B. thuringiensis Al Hakam represents intermediate relatedness.

A total of 256 candidate sequences were generated from subtractions by using genomic DNA from B. cereus ATCC 14579 as the driver (Table 2). Sequences that do not occur on plasmids pXO1 and pXO2 of B. anthracis were of primary interest, since some plasmid sequences are conserved in closely related Bacillus species. Sequences identical to pXO1 and pXO2 were eliminated by computer comparisons to published nucleotide sequences (see Materials and Methods). The remaining sequences were used to design oligonucleotide primers for PCR analysis, to determine their representation in the tester and driver genomes. PCR experiments with B. anthracis Ames and B. cereus ATCC 14579 DNA as a template identified 39 B. anthracis Ames-specific sequences (15% of the tester-specific candidates). Six of the tester-specific candidates (2%) did not amplify a product when DNA from the collection of non-anthrax-causing pathogens was used as a template. Similarly, 28 out of 48 sequences (58%) were absent from B. thuringiensis Al Hakam; 8 of these (16%) did not occur in the collection of closely related non-anthrax-causing pathogens (Table 2). Finally, 48 sequences each were generated from two separate subtractions by using genomic DNAs prepared from B. cereus 3A and B. thuringiensis Al Hakam as drivers. A total of 26 out of 48 sequences (54%) were present in B. anthracis Ames but absent from B. cereus 3A; 14 of these did not occur in the collection of closely related non-anthrax-causing pathogens (Table 2).

TABLE 2
Isolation of 28 B. anthracis-specific DNA signatures
from three SSH experiments

| | | | No. (%) |
|---|---|---|---|
| | | | of candidates |
| Tester | Driver | Candidate Sequence | remaining |
| B. anthracis | B. cereus | From SSH | 256 (100) |
| Ames | ATCC 14579 | Tester specific | 39 (15) |
| | | After screening with | 6 (2) |
| | | non-anthrax-causing | |
| | | Bacillus pathogens | |
| B. anthracis | B. thuringiensis | From SSH | 48 (100) |
| Ames | Al Hakam | Tester specific | 28 (58) |
| | | After screening with | 8 (17) |
| | | non-anthrax-causing | |
| | | Bacillus pathogens | |
| B. anthracis | B. cereus 3A | From SSH | 48 (100) |
| Ames | | Tester specific | 26 (54) |
| | | After screening with | 14 (29) |
| | | non-anthrax-causing | |
| | | Bacillus pathogens | |

It is crucial that DNA signatures intended for DNA-based detection of B. anthracis show no false-negative results with all isolates that might be encountered in environmental samples. All of the B. anthracis-specific candidates successfully amplified a PCR product when DNAs from the eight representatives of B. anthracis (Table 1) were used as templates (data not shown).

Distribution of tester-specific loci in B. cereus and B. thuringiensis.—Genomic variations within the collection of non-anthrax-causing pathogens were observed as the presence or absence of 39 tester-specific regions isolated from the subtraction in which genomic DNA from B. cereus ATCC 14579 was used as the driver and DNA from B. anthracis Ames was used as the tester (Table 3). The tester-specific primers are listed in Table 3 in decreasing order of the number of products amplified from this panel of non-anthrax-causing pathogens. Since, by definition, the driver will always be negative, there will be a maximum of nine loci possible in the 10 non-anthrax-causing Bacillus species. Similarly, the tester will always be positive, so that the minimum number of products possible will be one. The data in Table 3 show that seven primer sets are present in nine templates (excluding the driver), while six candidates (shown in bold type) are represented in B. anthracis only. The columns of data for non-anthrax-causing pathogens are arranged from left to right in Table 3 according to the number of tester-specific sequences seen in their respective genomes. The maximum number of candidates possible for each non-anthrax-causing Bacillus species is 39 for the tester (B. anthracis), while the minimum is 0 for the driver (B. cereus ATCC 14579). Strains that have more markers in common are suggested to be more closely related to B. anthracis. The data in Table 3 show that B. cereus 3A is the most closely related (31 out of 39 sequences), followed by B. cereus S2-8 and D17 (29 out of 39 sequences); as expected, B. thuringiensis ATCC 10792 is the least closely related (10 out of 39 sequences). The presence or absence of these loci is not correlated with the current species designations of B. cereus and B. thuringiensis. Table 4 shows data obtained with primers designed from the subtraction with the most closely related DNA as a driver. Far fewer primer candidates detect the non-anthrax-causing Bacillus species, with 14 (shown in bold type) being found only for B. anthracis Ames.

TABLE 3
TABLE 3 Presence or absence of B. anthracts Ames-specific nucleotide sequences in the genomes of 10
non-anthrax-causing Bacillus pathogens as determined by PCR with B. cereus ATCC 14579 as the driver

| | Presence (+) or absence (−) of B. anthracis Ames-specific nucleotide sequences in: | | | | | | | | | | |
|---|---|---|---|---|---|---|---|---|---|---|---|
| | | | | | | | | | | | B. |
| | B. | | | | | | | B. | | | cereus |
| | anthracis | B. | B. | B. | B. | B. | B. | cereus | B. | B. | ATCC |
| | Ames | cereus | cereus | cereus | thuringiensis | thuringiensis | thuringiensis | ATCC | cereus | thuringiensis | 14579 |
| Primer^{a} | (tester) | 3A | S2-8 | D17 | 97-27 | HD-571 | Al Hakam | 4342 | F1-15 | ATCC 10792 | (driver) |
| M. | + | + | + | + | + | + | + | + | + | + | − |
| Ctg015 | | | | | | | | | | | |
| R. | + | + | + | + | + | + | + | − | + | + | − |
| Ctg013 | | | | | | | | | | | |
| R. | + | + | + | + | + | − | + | + | + | − | − |
| Ctg048 | | | | | | | | | | | |
| R. | + | + | + | + | + | + | + | + | + | − | − |
| Ctg102 | | | | | | | | | | | |
| R. | + | + | + | + | + | + | + | + | + | + | − |
| Ctg107 | | | | | | | | | | | |
| R. | + | + | + | + | + | + | + | + | + | − | − |
| Ctg137 | | | | | | | | | | | |
| R. | + | + | + | + | + | + | + | + | + | + | − |
| Ctg173 | | | | | | | | | | | |
| M. | + | + | + | + | + | + | + | + | + | + | − |
| Ctg011 | | | | | | | | | | | |
| M. | + | + | + | + | + | + | + | + | + | − | − |
| Ctg013 | | | | | | | | | | | |
| M. | + | + | + | + | + | − | + | + | + | − | − |
| Ctg033 | | | | | | | | | | | |
| M. | + | + | + | + | + | + | + | + | − | − | − |
| Ctg038 | | | | | | | | | | | |
| M. | + | + | + | + | + | + | + | + | − | − | − |
| Ctg039 | | | | | | | | | | | |
| R. | + | + | + | + | + | + | + | + | − | − | − |
| Ctg070 | | | | | | | | | | | |
| R. | + | + | + | + | + | + | + | + | − | − | − |
| Ctg116 | | | | | | | | | | | |
| R. | + | + | + | + | + | + | + | + | + | − | − |
| Ctg153 | | | | | | | | | | | |
| R. | + | + | + | + | + | + | + | + | + | − | − |
| Ctg168 | | | | | | | | | | | |
| R. | + | + | + | + | + | + | + | + | − | − | − |
| Ctg018 | | | | | | | | | | | |
| R. | + | + | + | + | + | + | + | + | − | − | − |
| Ctg036 | | | | | | | | | | | |
| R. | + | + | + | + | + | + | + | + | − | − | − |
| Ctg087 | | | | | | | | | | | |
| M. | + | + | + | + | + | + | + | − | + | − | − |
| Crg025 | | | | | | | | | | | |
| R. | + | + | + | + | + | + | + | − | + | − | − |
| Crg079 | | | | | | | | | | | |
| R. | + | + | + | + | + | + | − | − | + | − | − |
| Ctg108 | | | | | | | | | | | |
| M. | + | + | + | + | + | + | + | − | − | − | − |
| Ctg003 | | | | | | | | | | | |
| R. | + | + | + | + | + | + | + | − | − | − | − |
| Ctg119 | | | | | | | | | | | |
| R. | + | + | + | − | + | + | + | + | − | − | − |
| Ctg156 | | | | | | | | | | | |
| M. | + | + | − | + | + | + | + | − | + | − | − |
| Ctg056 | | | | | | | | | | | |
| R. | + | + | + | + | + | − | − | − | − | − | − |
| Ctg100 | | | | | | | | | | | |
| M. | + | + | + | + | − | − | − | − | − | − | − |
| Ctg016 | | | | | | | | | | | |
| R. | + | + | + | − | − | − | − | + | − | − | − |
| Ctg185 | | | | | | | | | | | |
| R. | + | + | + | − | − | − | − | + | − | − | − |
| Crg197 | | | | | | | | | | | |
| M. | + | + | − | − | − | − | − | + | − | + | − |
| Ctg007 | | | | | | | | | | | |
| M. | + | − | − | + | − | − | − | + | − | − | − |
| Ctg031 | | | | | | | | | | | |
| M. | + | − | − | − | − | − | − | − | − | − | − |
| Ctg012 | | | | | | | | | | | |
| M. | + | − | − | − | − | − | − | − | − | − | − |
| Ctg032 | | | | | | | | | | | |
| M. | + | − | − | − | − | − | − | − | − | − | − |
| Ctg037 | | | | | | | | | | | |
| R. | + | − | − | − | − | − | − | − | − | − | − |
| Crg132 | | | | | | | | | | | |
| R. | + | − | − | − | − | − | − | − | − | − | − |
| Crg139 | | | | | | | | | | | |
| R. | + | − | − | − | − | − | − | − | − | − | − |
| Crg152 | | | | | | | | | | | |
| R. | + | − | − | − | − | − | − | − | − | − | − |
| Ctg177 | | | | | | | | | | | |

^{a} Bold rype indicates primers that are specific for B. anthracis.

TABLE
TABLE 4 Presence or absence of B. anthracis Ames-specific nucleotide sequences in the genomes of 10
non-anthrax-causing Bacillus pathogens as determined by PCR with B. Cereus 3A as the driver.

| | Presence (+) or absence (−) of B. anthracis Ames-specific nucleotide sequences in: | | | | | | | | | | |
|---|---|---|---|---|---|---|---|---|---|---|---|
| | B. | | | | | B. | | | B. | | B. |
| | anthracis | B. | B. | B. | B. | cereus | B. | B. | cereus | B. | cereus |
| | Ames | cereus | thuringlenis | thuringlenis | cereus | ATCC | thuringiensis | thuringiensis | ATCC | cereus | 3A |
| Primer^{a} | (tester) | D17 | HD-571 | Al Hakam | F1-15 | 4342 | 97-27 | ATCC 10792 | 14579 | S2-8 | (driver) |
| dhp77.46 | + | + | + | − | + | − | + | − | − | − | − |
| dhp73.18 | + | + | + | − | − | + | − | − | − | − | − |
| dhp73.03 | + | + | + | − | + | + | − | − | − | − | − |
| dhp77.38 | + | − | + | + | − | − | − | + | − | − | − |
| dhp73.04 | −− | − | − | + | − | − | + | − | − | − | − |
| dhp73.21 | + | + | − | − | − | − | − | − | − | − | − |
| dhp77.50 | + | + | − | − | − | − | − | − | − | | |
| dhp77.21 | − | − | − | − | + | − | − | − | − | − | − |
| dhp77.27 | − | − | − | − | − | − | − | − | − | − | − |
| dhp77.42 | − | − | − | + | − | − | − | − | − | − | − |
| dhp77.36 | + | − | − | − | − | − | − | − | − | − | − |
| dhp73.12 | + | − | − | − | + | − | + | − | − | − | − |
| dhp73.01 | + | − | − | − | − | − | − | − | − | − | − |
| dhp73.09 | + | − | − | − | − | − | − | − | − | − | − |
| dhp73.11 | + | − | − | − | − | − | − | − | − | − | − |
| dhp73.16 | + | − | − | − | − | − | − | − | − | − | − |
| dkp73.17 | + | − | − | − | − | − | − | − | − | − | − |
| dhp73.19 | + | − | − | − | − | − | − | − | − | − | − |
| dhp73.22 | − | − | − | − | − | − | − | − | − | − | − |
| dhp73.26 | + | − | − | − | − | − | − | − | − | − | − |
| dhp73.29 | + | − | − | − | − | − | − | − | − | − | − |
| dhp77.02 | − | − | − | − | − | − | − | − | − | − | − |
| dhp77.03 | + | − | − | − | − | − | − | − | − | − | − |
| dhp77.04 | + | − | − | − | − | − | − | − | − | − | − |
| dhp77.47 | + | − | − | − | − | − | − | − | − | − | − |
| dhp77.56 | + | − | − | − | − | − | − | − | − | − | − |

^{a} Bold type indicates primers that are specific for B. anthracis.

B. anthracis-specific DNA sequences and primers.—From the original collection of 352 fragments isolated from three subtractions, 28 were specific for B. anthracis and did not amplify a product with the near neighbors. All 93 tester-specific primers, some of which can be used to distinguish the B. cereus and B. thuringiensis strains used in this study, can be found at http:/bbrp.llnl.gov /htm/BAspc.htm. All primers successfully amplified a PCR product of the predicted size, showing no evidence of variations at these loci in the B. anthracis strains (data not shown). These 28 B. anthracis-specific candidates were used to screen DNAs from common enteric pathogens—Yersinia pestis, Y. pseudotuberculosis, Y. enterocolitica, and Escherichia coli—species that may be encountered in environmental samples in suspected anthrax cases (soil, air, human, and bovine samples). All 28 candidates failed to amplify a product from these bacterial DNAs (data not shown). The average G+C content of these sequences was 35% (28 to 44%), a value consistent with that of B. anthracis.

All but 1 of the 28 B. anthracis-specific DNA sequences can be mapped to the 5.23-Mb genome of B. anthracis A2012 (GenBank accession number NC_{—}003995). BLASTX analysis showed that 24 of the remaining 27 DNA sequences map to the open reading frames defined therein, while the remainder map to intergenic regions. The coordinates for nucleotide identity to B. anthracis A2012, open reading frame identity, and gene identity are listed in Table 5. BLASTX identities with E values of less than 10⁻³ are also listed. BLAST data for the candidates from the B. cereus ATCC 14579 subtraction show that three of the six DNA sequences have no previously ascribed function, based on similarity searches of the GenBank database. One of the remaining three shows identity with the S-layer protein of B. anthracis, and the other two show identity with a hypothetical phage protein from Streptococcus pyogenes and a galactosyl transferase-related protein from Clostridium acetobutylicum. BLAST data for the candidates from the B. thuringiensis Al Hakam subtraction show that four of the eight DNA sequences have no previously ascribed function. The remaining four share sequence identity with a hypothetical protein in B. halodurans, a penicillin binding protein of B. cereus, a putative phage terminase of C. perfringens, and a cytosine-specific methyltransferase of B. halodurans. BLAST data for the candidates from the B. cereus 3A subtraction show that 9 of the 14 DNA signatures have no previously ascribed function. The remaining five show sequence identity with the Staphylococcus aureus terminase large subunit, a hypothetical protein from the nanH region and an ATP binding cassette transporter of C. perfringens, a glucosamine synthetase of B. subtilis, and an unknown conserved protein of B. halodurans.

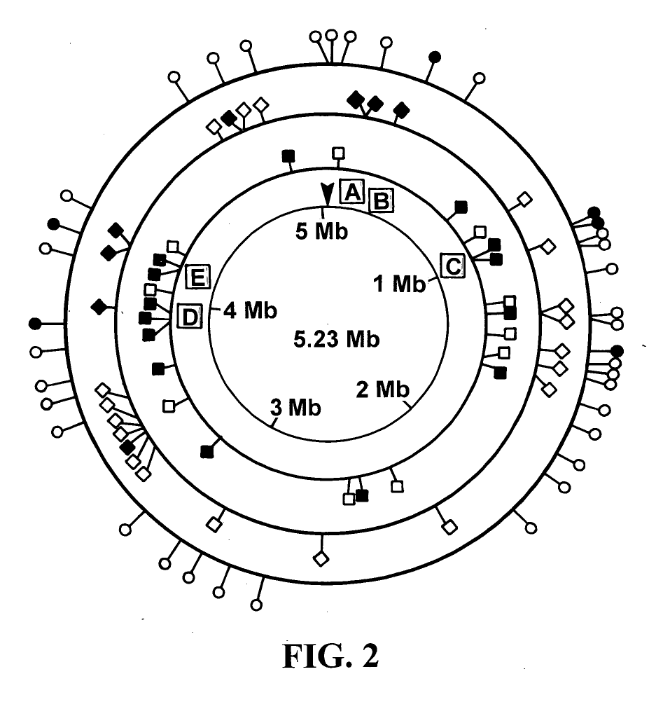

Referring now to FIG. 2, a visualization of the distribution of the tester-specific sequences for each of the three subtractive hybridization experiments is shown. FIG. 2 provides a Graphic representation of the locations of the tester-specific clones on the B. anthracis A2012 genome. The outermost circle maps the locations of the clones isolated from the subtraction by using DNA from B. cereus ATCC 14579 as the driver (circles), the second circle maps clones isolated from the subtraction by using DNA from B. thuringiensis Al Hakam as the driver (diamonds), and the third circle maps clones isolated from the subtraction by using DNA from B. cereus 3A as the driver (squares). The innermost circle shows the coordinates of the B. anthracis genome (5.23 Mb); the arrow shows the location of the first nucleotide. The sequences that are seen in B. anthracis but that are not seen in any non-anthrax-causing pathogens are represented by closed symbols. Gray boxes A to E, adjacent to the innermost circle, indicate the five regions that contain more than two B. anthracis-specific DNA sequences within 50 kb of each other; these represent putative B. anthracis-specific genomic islands.

There are five regions that have more than two B. anthracis-specific loci that lie within 50 kb of each other, suggesting genomic islands that are found only in B. anthracis. Similar genomic islands have been observed when genomes of different strains of the same bacterial species have been compared (e.g., Y. pestis and E. coli.

Multiplex PCR analysis for the simultaneous detection of four B. anthracis-specific loci.—Multiplex PCR is a powerful tool for the simultaneous detection of multiple loci within bacterial genomes. The simultaneous detection of four separate loci (A, C, D, and E) on the B. anthracis genome was achieved here by selecting primers (shown in bold type in Table 5) that target these loci while providing sufficient size discrimination for resolution by gel electrophoresis on 4% agarose (163, 133, 196, and 241 bp, respectively). An internal positive control (288 bp) was designed from a region of the 23S gene conserved in Bacillus species (see Material and Methods) and confirmed the integrity of the DNA template.

Referring now to FIG. 3, a multiplex analysis of four separate loci on the B. anthracis genome is shown. FIG. 3 shows that the Bacillus 23S control primer yielded a 288-bp PCR product for all DNA templates (10 ng) tested.

One internal positive control yielded a predicted product of 288 bp. The DNA templates used for the multiplex analysis were as follows: lanes 1, 12, and 13, size markers as indicated; lane 2, B. cereus ATCC 14579; lane 3, B. cereus ATCC 4342; lane 4, B. cereus D17; lane 5, B. cereus 3A; lane 6, B. cereus S2-8; lane 7, B. cereus F1-15; lane 8, B. thuringiensis 97-27; lane 9, B. thuringiensis Al Hakam; lane 10, B. thuringiensis ATCC 10792; lane 11, B. thuringiensis HD-571; lane 14, B. anthracis G3; lane 15, B. anthracis G20; lane 16, B. anthracis G25; lane 17, B. anthracis G29; lane 18, B. anthracis G38; lane 19, B. anthracis G62 (Ames); lane 20, B. anthracis G67; lane 21, B. anthracis G77 (Vollum); lane 22, B. anthracis G80; lane 23, B. anthracis G87; and lane 24, no-template negative control.

Furthermore, the four predicted B. anthracis-specific bands were seen for all strains (FIG. 3, lanes 13 to 23) and were absent from B. cereus and B. thuringiensis strains (lanes 2 to 11). This multiplex PCR was capable of detecting all four loci with as little as 100 pg of template DNA (data not shown). FIG. 3 shows Multiplex analysis of four separate loci on the B. anthracis genome. Four B. anthracis-specific primers yielded predicted products of 133, 163, 196, and 241 bp. One internal positive control yielded a predicted product of 288 bp. The DNA templates used for the multiplex analysis were as follows: lanes 1, 12, and 13, size markers as indicated; lane 2, B. cereus ATCC 14579; lane 3, B. cereus ATCC 4342; lane 4, B. cereus D17; lane 5, B. cereus 3A; lane 6, B. cereus S2-8; lane 7, B. cereus F1-15; lane 8, B. thuringiensis 97-27; lane 9, B. thuringiensis Al Hakam; lane 10, B. thuringiensis ATCC 10792; lane 11, B. thuringiensis HD-571; lane 14, B. anthracis G3; lane 15, B. anthracis G20; lane 16, B. anthracis G25; lane 17, B. anthracis G29; lane 18, B. anthracis G38; lane 19, B. anthracis G62 (Ames); lane 20, B. anthracis G67; lane 21, B. anthracis G77 (Vollum); lane 22, B. anthracis G80; lane 23, B. anthracis G87; and lane 24, no-template negative control.

Discussion—. anthracis is a potent mammalian pathogen and bioterrorist agent. This pathogenic Bacillus species shares so much genetic material with B. cereus and B. thuringiensis that its discrimination from the other species can be problematic. All three Bacillus species are prevalent in many environments, and it is important to define the genetic differences specific to B. anthracis in order to design specific DNA-based identification protocols. We surmised that the most efficient approach to finding B. anthracis-specific DNA sequences would be to find the Bacillus species that are most closely related to B. anthracis and then to compare their genomes in vitro by using SSH.

AFLP analysis was used to reveal genetic diversity among non-anthrax-causing Bacillus strains and to determine which are most closely related to the highly pathogenic B. anthracis. The phylogenetic tree shown in FIG. 1 indicates that B. cereus 3A, B. cereus S2-8, and B. thuringiensis 97-27 are the strains most genetically similar to B. anthracis of the 10 strains examined. B. cereus ATCC 4342 was shown previously by multienzyme electrophoresis and AFLP analysis to be very closely related to B. anthracis. The AFLP analysis presented here shows that seven strains are even more closely related to B. anthracis. The relationships do not correlate with the species designation for B. cereus or B. thuringiensis, providing another example of how the species designations for B. cereus and B. thuringiensis appear to cross the boundaries of various phylogenetic analyses. It may be necessary to develop new criteria for species designations within this group, as information about more Bacillus genomes becomes available.

Although AFLP analysis provides a sensitive method for defining the genetic relationships between bacterial genomes, it provides no information regarding the genetic rearrangements responsible for these differences. Such information can be attained by in vitro genome comparison by SSH, a proven, efficient method for the identification of nucleotide sequences that differ between two genomes. The complementary techniques of AFLP analysis and SSH provide a powerful means of defining accurate phylogenetic models and characterizing their underlying genetic components. Three subtractions were performed to identify B. anthracis-specific nucleotide sequences that were absent from the non-anthrax-causing Bacillus species. B. cereus ATCC 14579 represents the most distantly related strain in this study. B. cereus 3A was chosen because it represents one of the closest relatives of B. anthracis. The third strain, of intermediate relatedness, was B. thuringiensis Al Hakam. It would be expected that strains with more sequence identity to B. anthracis (in this situation, B. cereus 3A) would yield more sequences that would not be found in the closely related B. cereus and B. thuringiensis. Indeed, the subtraction with B. cereus 3A yielded 29% B. anthracis-specific sequences, compared to 17 and 2% for B. thuringiensis Al Hakam and B. cereus ATCC 14579, respectively (Table 2), confirming the prediction from AFLP analysis.

The genetic diversity demonstrated within this strain collection by AFLP analysis was mirrored by the results of subsequent PCR analysis. Bacterial evolution is driven by rearrangements of large genomic islands associated with lateral gene transfer. Analysis of the G+C contents of the difference products reveals an average of 35.4%, typical of B. anthracis. This finding suggests that any lateral gene transfer has ameliorated the G+C content over a long period of evolution or has been received from species with a similar G+C content. The presence or absence of each of the tester-specific sequences in the non-anthrax-causing pathogens was determined by PCR amplification with primers designed for the difference products. If the number of loci shared by B. anthracis indicates relatedness among the whole panel of non-anthrax-causing Bacillus strains, then B. cereus 3A is the most closely related (Table 3). This result is in agreement with the phylogenetic analysis shown in FIG. 1. The phylogenetic tree shows two pairs of isolates that cannot be distinguished by AFLP analysis: Bacillus strains Vollum and Ames and B. cereus strains 3A and S2-8. SSH with B. cereus ATCC 14579 as a driver yielded two primer sets (M.Ctg056 and M.Ctg007) that can distinguish B. cereus 3A from S2-8 (Table 3). Primers from the same subtraction cannot distinguish B. thuringiensis HD-571 from Al Hakam. Table 4 demonstrates that the number of loci that are shared between the non-anthrax-causing Bacillus species and B. anthracis decreases when the driver strain is much more closely related to B. anthracis. This subtraction also yielded two primer sets (dhp73.03 and dhp73.04) that can distinguish B. thuringiensis HD-571 from Al Hakam, which are indistinguishable by the primer sets listed in Table 3.

The most important criteria for effective DNA signatures are the absence of false-positive results with closely related organisms and their representation in all isolates of the target (i.e., no false-negative results). Given the monomorphic nature of the B. anthracis genome, it was not surprising to find that there was no variation in the signatures within the strains of the collection. Twenty-eight B. anthracis-specific candidates isolated in these experiments fulfilled these criteria and are listed in Table 5. None amplified a PCR product from any of the non-anthrax-causing Bacillus pathogens used in this study (no false-positive results). We were also able to exploit a collection of genetically distinct and geographically diverse isolates of B. anthracis. The twenty-eight DNA signatures amplified a PCR product of the predicted size for every isolate. A comparison of the DNA sequences against the completed genomes of B. halodurans and B. subtilis and the unfinished Bacillus genomes showed no significant sequence identity.

The non-anthrax-causing Bacillus species described here are so closely related to B. anthracis that they would be highly likely to give false-positive results in DNA-based identification assays based on chromosomal loci. The isolation of multiple B. anthracis-specific chromosomal regions allowed the development of a single multiplex assay for the rapid and highly specific detection of B. anthracis. The DNA signatures presented here have the advantage over previous detection methods that require time-consuming analysis, are prone to false-positive results, and are based on few nucleotide differences at a single chromosomal locus.

BLAST analysis of the nucleotide sequences of these DNA signatures shows that many are not represented in current DNA databases, other than the previously reported B. anthracis A2012 genome (Table 5). The strongest identity seen was to the penicillin binding protein of B. cereus (dhp64.177; E value, 10⁻⁶⁷). R.Ctg122 showed identity to the S-layer protein of B. anthracis. This cell surface protein is also seen in some isolates of B. cereus and B. thuringiensis. However, there is sufficient nucleotide sequence divergence at the oligonucleotide primer binding sites to allow for successful discrimination of B. anthracis from the other two species.

The genomes of several strains of B. anthracis are currently being sequenced, and these data will be extremely useful for strain attribution in forensic analyses. We envisage that these DNA signatures can be used for real-time specific detection of B. anthracis, the source of which may then be attributed by monitoring the small nucleotide differences identified by these sequencing projects. The B. anthracis-specific DNA sequences identified in this work provide the largest collection of chromosomal markers that distinguish B. anthracis from other closely related Bacillus species. There are five regions in the B. anthracis genome where several of the specific DNA sequences are located within 50 kb of each other. Such genomic islands may define B. anthracis as a species and distinguish it from the closely related species B. cereus and B. thuringiensis. Future detailed analysis of these B. anthracis-specific regions may ultimately identify chromosome-encoded virulence factors, provide starting points for possible vaccine candidates, and help to reveal the mode of pathogenicity of this important pathogen.

Preparation of genomic template DNA for PCR—The bacterial strains used for genomic DNA preparations, and their geographic origin are listed where known in Table 1. DNA was extracted from cell pellet from overnight cultures grown in L-broth using the MasterPure DNA Purification Kit from Epicentre (Madison, Wis.) according to the manufacturer's instructions. Genomic DNA prepared from enteric bacteria using Wizard Genomic DNA Preps (Promega; Madison, Wis.) according to manufacturer's instructions. Human genomic DNA was purchased from Clontech (Palo Alto, Calif.) and bovine genomic DNA was purchased from Novagen (Madison, Wis.). Soil DNA was extracted using UltraClean Soil DNA Kit from MoBio (Solana Beach, Calif.) according to the manufacturer's instructions. DNA isolated from organisms present in the air was prepared from filters as previously described. Both soil and air filter DNA was prepared from samples that originated in Livermore, Calif., After ethanol precipitation, all genomic DNA was dissolved in 10 mM Tris HCl, pH8.0, to a concentration of approximately 0.2 μg/ml.

Polymerase Chain Reactions—Oligonucleotide primers were designed from the putative tester-specific sequences and were supplied by Sigma-Genosys (The Woodlands, Tex.) or Invitrogen (Carlsbad, Calif.). The primers were designed using Primer3 software, and have a melting temperature of >65° C., contain no more than three identical consecutive nucleotides and possess a two nucleotide 3′ GC clamp. The primers were initially screened against genomic DNAs from both the tester and the driver DNA. To determine whether a primer pair was tester-specific, 1 ng each of the tester and driver DNA was used as template in PCR reactions using Accuprime polymerase and primers at 10 μM (Invitrogen; Carlsbad, Calif.) with the following cycling parameters: 94° C. for 15 seconds, 65° C. for 15 seconds, 72° C. for 30 seconds for 32 cycles. The products were visualized on 1.5% agarose gel run in 0.5×TBE; if a product was present with tester DNA as template and absent with the driver that sequence was designated tester-specific. The tester-specific oligonucleotides were then used to prime PCRs using the B. anthracis, B. cereus and B. thuringiensis strains listed in Table 1, and the same reaction conditions described above. The integrity of the genomic DNA template was tested in all PCRs using primers specific for a region of the 23S gene conserved in Bacillus species: 5′-CTACCTTAGGACCGTTATAGTTAC (SEQ ID NO: 35), and 5′-AGGTAGGCGAGGAGAGAATCC (SEQ ID NO: 36). Multiplex PCR reactions were performed as above, except that template DNA was added to a final concentration of 10 ng/μl. The primers used are listed in Table 4 and were used at the following concentrations: 23S primers at 3 μM (288 bp), dhp73.017 (241 bp) at 20 μM, dhp73.019 (196 bp) at 10 μM, dhp61.183 (163 bp) at 5 μM, and dhp77.002 (133 bp) at 20 μM.

Descriptions of the set of twenty eight chromosomal DNA signatures are provided in Table 6 and are included as part of this Application.

TABLE 6
Set of twenty eight chromosomal DNA signatures.

SEQ ID NO:

| | | |
|---|---|---|
| 1. | R.Ctg132 | ACCTTTTGCTAAAGTGATTTCACCTTTTGCATTAATTTTTTC |
| | | TGCAGCTGGCGTTACAAATTCAATACCCTCTGGTGAAACAAC |
| | | TTCAGT |
| 2. | R.Ctg139 | CAAAGTGGCAAAAGGGGAATTTAGAATATGCTAGTTTCCGTT |
| | | CTATGACAATCGATGAATTTATTGATCCGTTGPCTTTTCTTA |
| | | AAAAGATTGCTTCTTTGTTTGAATTAGAAATTCAATATCGTG |
| | | CTGGAGTTGTAGGCTCTCAAGTTGTTGGGCG |
| 3. | R.Ctg152 | TCCCAATACATATGAGCGATTCGCCTTTATAAACGACGTATT |
| | | CCTTTGAACTCGTTATGACACTCATTACTCAACTCCCCTTTT |
| | | CTACTAAAATAGCGTTTTTGTTTGGTTTTTTTCTTCACATAA |
| | | TCCGTCCTATTTGATTTTTACATACCACC |
| 4. | R.Ctg177 | TTGGATCAGCGTTTCTGAATTCAGCTAATAATACAACGTCTT |
| | | CTGGATCTAATGAATATAATCTTGTTAATGCAAGTGGAGTTG |
| | | ATATAAACGTAGCACAGAAACTTAGGGGAATACGTAATTATA |
| | | GCATTAAAGTAGATGGAATTGAGCGATATGGGGA |
| 5. | M.Ctg032 | GGCCCCATAACTTGATTTCTCTTCAATATGAAAGTTGCTGCC |
| | | TCATCAACATTTTCGAAAATAAAATCTTCATCATAAATCGTA |
| | | TGACAACCTTCCCACCTTATAATTATAGGAAAGGCACCCGAC |
| | | GCCATTCCTTCAGCTGGAGCAAGAT |
| 6. | M.Ctg037 | GAAATGAATGCAGAAAAATTAGAGCTTGCGAAAAATCCACCT |
| | | GACAGTGCAGCAGTACAAGAAATATTTGCTCAATTAGAAGGT |
| | | TGGGGTAAAGCTATTGCTAAAGGTGGAGAAGAAGGTAAACAA |
| | | GCATTTGTA |
| 7. | dhp61.159 | AATTCTCATCCTACAGCATCTCCAATAATATATATTCCAACC |
| | | AAAAGATGACCATGTTACGGTTATAAAATACCAATCTATTAT |
| | | TTCATCGCGTCCTTCACAACATCTTGAATCATAACGACTTCT |
| | | GTTTTCATATCAACAGAGGGTGCTTCTTCA |
| 8. | dhp61.178 | GGTGGGTAAATGATGAGTGTTTCAACAAAAATTAATCAGCTA |
| | | ATAAGTCTTAGAGAAAAAATCGCTAAAGATTTAGGGTTTTAT |
| | | GTTAACTTTGATTTAGCGAATTCAAGTTGGTCATACTCTAAA |
| | | AAGGCTGGTCAAATCAAAAA |
| 9. | dhp61.181 | TGTGGAAGAATCCACTGTCCAAGAACGTGAGCGTGTGCTATG |
| | | GGATGAAATGGCTTCACCAATAGGATGGACTCCTGTTACTGG |
| | | GCAAGTTGAGGAAATGAAAGAGACAGA |
| 10. | dhp61.183 | GAAGGACGATACAGACATTTATTGGGAACTACACATGCACTA |
| | | GGGTCATTGTTCATTAGCTCATCGATTGCATTATTAACAACG |
| | | GCTTTTATTGTCCTTACGCATTTATCTAGCTTTATGAACAAT |
| | | TACTATTTTTGGATAGGGCTTGCTATTCAACTTGCGGT |
| 11. | dhp64.177 | TCGCAAGATAAATGGGAAGACGAAAAGCGGGCTGGACAATAT |
| | | TCTCCGACATACCAGGGACAGCTTCCAAGTGAAATGGTAAAT |
| | | GTGATTGGTACGGGAGGA |
| 12. | dhp64.188 | TTGGTAAAGTAGAAGCATCTTGGTTAAGAGCAGGATTACTCA |
| | | TGGGTATGACGATAAAAACAAGCAATACAAATGAACATTTGC |
| | | ATATGGAAAACCAGGTGATGCGTTTTGTAAACCAAGGGTCAG |
| | | ATAAGATGG |
| 13. | dhp64.202 | ATATGTGGTTAGGAGCCAATGAAGGTGATGGTGACTTCTTTG |
| | | TAGATAAACATACTTTACAAAAAAGCTTGTACAAGCCAGCTT |
| | | CTGATTGGGAATGGTGGAGAGGAAAACGTCAGGTGATTATTG |
| | | GACTCGATTTGT |
| 14. | dhp64.208 | ACTACTCGACAAGGACGTTTGTATTTCCGGCTGTCAGTGTCG |
| | | GCGCGCCGCATCAAAGATACCGGACATTTATTGTTGGCCACT |
| | | CCAACGACAAGTCAAAATTACAAACCGATCCGAGAGTTGTGC |
| | | CCTTCAGAAGCAAATGGGAAACATGG |
| 15. | dhp77.002 | TGATATTTATGACCAAGATTCAATATACGAATATAATCCCAA |
| | | AAGAGCCAATCATGCAATGGAGTTCATTGAGAATTTTTGCAA |
| | | GCATTCAAAGGGGAAATGGGCTGGAAAACCTATTGACCTTGA |
| | | GCTATGGC |
| 16. | dhp77.003 | CCAGGTCCATCCTATGTTGCAGTAGAAACGTATGTTACAGTA |
| | | GAAGCGTATTTTAGCAAACGTGAGTACGCACTACCTCCAGCT |
| | | AGATACTATCATCAAGAAACGCGAGATGGGATGATATATTCT |
| | | GGTTACCTTACCAGACATAGTTATGCATCAACGGGAGACACG |
| | | CGGATTGTAGT |
| 17. | dhp77.004 | CACGTGAGTTTACACCATACGCGGCGAACGCAAAGCGTTCAA |
| | | GATTAGAACAAGAAGTGAATATTGATTTTTATAAAAGAGAAA |
| | | TTTTCACATATGCAGATGCTTGTATCGTCACAGTAAGGATTA |
| | | CAAATCCAGATGGCTCAACTGAGTATCAAAAAGGAGAAATAA |
| | | GTACAGAAAACATTGTATGTACTAATATCGTATGGGGCGAGG |
| | | ATGAAGTTTCATTCGAAATGAGAGCAAGTGCTAG |
| 18. | dhp77.047 | GGAACAACTTTCCCACAAGCTGAACTTGAGTAGAAGCCAGAT |
| | | CAAAAACTGGGAAACTGATCGGTATCAGCCAGATATAGATAC |
| | | GTTGGTTATCATCGCCTCCTTCTTCAATGTTTCGGTAGACGC |
| | | GCTTATTGGGTTCACGAATGA |
| 19. | dhp77.056 | AGGAACGTGCGACGACTAGCTGAAAAGCGTAGGGTGTAAGCC |
| | | AATGACATCCGAAATGGGGAGCATCTTATATAAAAGATGATG |
| | | ATATAGTCTGGTCTGTATAGTGATATACAGAAGTTCATAAGA |
| | | GAACTGGCAGGATGTTGCGAGTCCTGTTGAACATATCGGGTG |
| 20. | dhp73.001 | TTTCTTATCCAACAGGTGTATGCTTATGTTTTATAAAAAAGT |
| | | TTGATCATAGCTTATTTACAATTCTTTGATTAAAGAGATAAA |
| | | GCATCGCACCTTTTTTATAAACGTCTTAACTTTATTTCAAAA |
| | | CTACACTATATTTACTAACCTACTAATCTCCTACTCAACCGT |
| | | AACAGACTTAGCTAAGTTACGTGGCTTATCAACGTCACACTC |
| | | GC |
| 21. | dhp73.009 | TCAAACGTTGCCGAATAAGCCAATTCATTTAGATGCGTTACT |
| | | TAATAAACTATTTTTAGGTAATACGAAGGACCGTATTAAAGC |
| | | AGCAATTGAGTTAATTGAGTGGGTGCTTCATAAATAATATTG |
| | | TCTAATATTGTCGGTAAATCGATATGTTGTGTCGAATCGTC |
| 22. | dhp73.011 | TTGGCATTGTGTTAGATGAGGGGTATTTCTATGATGAATTAA |
| | | CATTGAAAGAAATGAAAAATGTGATTGCCCCTTCTTATACTG |
| | | ACTGGGATGAAGCAGTCTTCTTATCTTATATTAAACAATTTA |
| | | ATTTAAATCTAAGACAAAAAATTTCTACTCTATCTAAAGGGA |
| | | TGCGGATGAAATTTGCGGTTGCTTTGGCTTTATCACA |
| 23. | dhp73.016 | CAGAGCGTTTCTTAAATGAAGAGGATTTAATTGGAGAGCATT |
| | | TAGAGAGAGAAGATATTGATATCACATATTTAGATGACACGA |
| | | TGTATATATTGTTGCCATATAAGAATGATCAAGAATTAAAAC |
| | | AGAAAAAGTTAGATAAAATCCAAATTGAGGCATATGATGAA |
| 24. | dhp73.017 | AAAGGCGGTTTAGAATTTGGACCGTATCAAAATTTAAAACAA |
| | | GTTGAGTCGATAGCAGAAAAGCCAATTCAAGCATTTCAAGTT |
| | | TTAAATACCATTCTTGAGAAAATGGAAGAGAAAATGTTCTAT |
| | | ATGAAGGAAAGACATTATACAAACGTTGTAGAAACAAATATA |
| | | AAAGAGCGTCATTTCATTATAGTTGATGAAGGGGCTGAACTT |
| | | TGTCCTGATAAAAGCATGGGTAAAGAGCAGCA |
| 25. | dhp73.019 | TGTAAATGAACGCCTTGACCAATCACCAGCAGGGAATTCAGT |
| | | TTGTAGATTTTCTGTCCATGATCCGTTGATAAATTGGAATAC |
| | | AAGAAAAGTAACGTAAACATCATTTTGAAGATCGACACTGAA |
| | | TGTCATGTCTTTACCTTTTTCAAAATTACCCATCTTAGTATT |
| | | ATCTAAATGGAATTGATAGAAGGAGTCGG |
| 26. | dhp73.022 | AAACAATGTTGGATGGTAAATGGCATTACTTCCACGGCAAAT |
| | | TAAAAGTTGTGGATGGGAAAGTGCGTGTTTACTTTGGGATGT |
| | | TGAAGACGGGAAATGTAAAAGGTGCTAAAACAAGAATAACCG |
| | | GAGTGAAATTTGCGAATGG |
| 27. | dhp73.026 | CCGAGCTTTAATGGAAATGGAGAATTATTATATGATTTTAGG |
| | | GATTTCATATTTTAATATTTTTTGTATTGTAGTGATTATCTT |
| | | GTTTTTCTTTATTTTTTGGAGATTGCATGTATGGTATAACAA |
| | | GAAACACAATGTTCCTAAGGTTTTTCAATGGTTTCCTAGAAA |
| | | ATGGGGTAAAAGAAAAGTGTCAGAGCATTTATCACAATTGAA |
| | | TGAAAAGCTAGAGGCTGAAGGTAAAGG |
| 28. | dhp73.029 | GGAGAAACAGATAGTGGTGAAAGCTGGGAGTATATTTACTTC |
| | | TTAGATGAAATAAAGCATCAAGCCATTAGTTTAAGTGTCTTT |
| | | AATAGATTATTAGATTATGAAGAAGGAAATCTAATACAAGGC |
| | | TTTAGAGT |

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

A paper copy disclosing the nucleotide and/or amino acid sequences is attached as the “Sequence Listing.” In addition, a copy of the “Sequence Listing” is being submitted in computer readable form. The computer readable form is a copy of the “Sequence Listing.”

The invention claimed is:

1. A method of detecting Bacillus anthracis, comprising the steps of:
selecting at least one individual locus on a Bacillus anthracis chromosome,
using a multiplex polymerase chain reaction analysis of said individual locus on a Bacillus anthracis chromosome to produced at least one chromosomal DNA signature, and
using said at least one chromosomal DNA signature to detect genetically Bacillus anthracis.

2. The method of detecting Bacillus anthracis of claim 1 wherein said step of using a multiplex polymerase chain reaction analysis of said individual locus on a Bacillus anthracis chromosome to produced at least one chromosomal DNA signature comprises using a multiplex polymerase chain reaction analysis of said individual locus on a Bacillus anthracis chromosome to produced a set of twenty eight chromosomal DNA signatures.

3. The method of detecting Bacillus anthracis of claim 1 wherein said at least one chromosomal DNA signature comprises:
(SEQ ID NO: 1)
ACCTTTTGCTAAAGTGATTTCACCTTTTGCATTAATTTTTTCTGCAGCTG
GCGTTACAAATTCAATACCCTCTGGTGAAACAACTTCAGT.

4. The method of detecting Bacillus anthracis of claim 1 wherein said at least one chromosomal DNA signature comprises:
AAGTGGCAAAAGGGGAATTTAGAATATGCTAGTTTCCGTTCTATGACAAT
CGATGAATTTATTGATCCGTTGTCTTTTCTTAAAAAGATTGCTTCTTTGT
TTGAATTAGAAATTCAATATCGTGCTGGAGTTGTGGCTCTCAAGTTGTTG
GGCG. (residues 3-157 of SEQ ID NO: 2)

5. The method of detecting Bacillus anthracis of claim 1 wherein said at least one chromosomal DNA signature comprises:
(SEQ ID NO: 3)
TCCCAATACATATGAGCGATTCGCCTTTATAAACGACGTATTCCTTTGAA
CTCGTTATGACACTCATTACTCAACTCCCCTTTTCTACTAAAATAGCGTT
TTTGTTTGGTTTTTTTCTTCACATAATCCGTCCTATTTGATTTTTACATA
CCACC.

6. The method of detecting Bacillus anthracis of claim 1 wherein said at least one chromosomal DNA signature comprises:
(SEQ ID NO: 4)
TTGGATCAGCGTTTCTGAATTCAGCTAATAATACAACGTCTTCTGGATCTA
ATGAATATAATCTTGTTAATGCAAGTGGAGTTGATATAAACGTAGCACAG
AAACTTAGGGGAATACGTAATTATAGCATTAAAGTAGATGGAATTGAGC
GATATGGGGA.

7. The method of detecting Bacillus anthracis of claim 1 wherein said at least one chromosomal DNA signature comprises:
(SEQ ID NO: 5)
GGCCCCATAACTTGATTTCTCTTCAATATGAAAGTTGCTGCCTCATCAACA
TTTTCGAAAATAAAATCTTCATCATAAATCGTATGACAACCTTCCCACCTT
ATAATTATAGGAAAGGCACCCGACGCCATTCCTTCAGCTGGAGCAAGAT.

8. The method of detecting Bacillus anthracis of claim 1 wherein said at least one chromosomal DNA signature comprises:
(SEQ ID NO: 6)
GAAATGAATGCAGAAAAATTAGAGCTTGCGAAAAATCCACCTGACAGTG
CAGCAGTACAAGAAATATTTGCTCAATTAGAAGGTTGGGGTAAAGCTATT
GCTAAAGGTGGAGAAGAAGGTAAACAAGCATTTGTA.

9. The method of detecting Bacillus anthracis of claim 1 wherein said at least one chromosomal DNA signature comprises:
(SEQ ID NO: 7)
AATTCTCATCCTACAGCATCTCCAATAATATATATTCCAACCAAAAGATG
ACCATGTTACGGTTATAAAATACCAATCTATTATTTCATCGCGTCCTTCAC
AACATCTTGAATCATAACGACTTCTGTTTTCATATCAACAGAGGGTGCTTC
TTCA.

10. The method of detecting Bacillus anthracis of claim 1 wherein said at least one chromosomal DNA signature comprises:
(SEQ ID NO: 8)
GGTGGGTAAATGATGAGTGTTTCAACAAAAATTAATCAGCTAATAAGTCT
TAGAGAAAAAATCGCTAAAGATTTAGGGTTTTATGTTAACTTTGATTTAG
CGAATTCAAGTTGGTCATACTCTAAAAAGGCTGGTCAAATCAAAAA.

11. The method of detecting Bacillus anthracis of claim 1 wherein said at least one chromosomal DNA signature comprises:
(SEQ ID NO: 9)
TGTGGAAGAATCCACTGTCCAAGAACGTGAGCGTGTGCTATGGGATGAA
ATGGCTTCACCAATAGGATGGACTCCTGTTACTGGGCAAGTTGAGGAAAT
GAAAGAGACAGA.

12. The method of detecting Bacillus anthracis of claim 1 wherein said at least one chromosomal DNA signature comprises:
(SEQ ID NO: 10)
GAAGGACGATACAGACATTTATTGGGAACTACACATGCACTAGGGTCAT
TGTTCATTAGCTCATCGATTGCATTATTAACAACGGCTTTTATTGTCCTT
ACGCATTTATCTAGCTTTATGAACAATTACTATTTTTGGATAGGGCTTGC
TATTCAACTTGCGGT.

13. The method of detecting Bacillus anthracis of claim 1 wherein said at least one chromosomal DNA signature comprises:
(SEQ ID NO: 11)
TCGCAAGATAAATGGGAAGACGAAAAGCGGGCTGGACAATATTCTCCGA
CATACCAGGGACAGCTTCCAAGTGAAATGGTAAATGTGATTGGTACGGG
AGGA.

14. The method of detecting Bacillus anthracis of claim 1 wherein said at least one chromosomal DNA signature comprises:
(SEQ ID NO: 12)
TTGGTAAAGTAGAAGCATCTTGGTTAAGAGCAGGATTACTCATGGGTATG
ACGATAAAAACAAGCAATACAAATGAACATTTGCATATGGAAAACCAG
GTGATGCGTTTTGTAAACCAAGGGTCAGATAAGATGG.

15. The method of detecting Bacillus anthracis of claim 1 wherein said at least one chromosomal DNA signature comprises:
(SEQ ID NO: 13)
ATATGTGGTTAGGAGCCAATGAAGGTGATGGTGACTTCTTTGTAGATAAA
CATACTTTACAAAAAAGCTTGTACAAGCCAGCTTCTGATTGGGAATGGTG
GAGAGGAAAACGTCAGGTGATTATTGGACTCGATTTGT.

16. The method of detecting Bacillus anthracis of claim 1 wherein said at least one chromosomal DNA signature comprises:
(SEQ ID NO: 14)
ACTACTCGACAAGGACGTTTGTATTTCCGGCTGTCAGTGTCGGCGCGCCG
CATCAAAGATACCGGACATTTATTGTTGGCCACTCCAACGACAAGTCAA
AATTACAAACCGATCCGAGAGTTGTGCCCTTCAGAAGCAAATGGGAAAC
ATGG.

17. The method of detecting Bacillus anthracis of claim 1 wherein said at least one chromosomal DNA signature comprises:
(SEQ ID NO: 15)
TGATATTTATGACCAAGATTCAATATACGAATATAATCCCAAAAGAGCC
AATCATGCAATGGAGTTCATTGAGAATTTTTGCAAGCATTCAAAGGGGAA
ATGGGCTGGAAAACCTATTGACCTTGAGCTATGGC.

18. The method of detecting Bacillus anthracis of claim 1 wherein said at least one chromosomal DNA signature comprises:
(SEQ ID NO: 16)
CCAGGTCCATCCTATGTTGCAGTAGAAACGTATGTTACAGTAGAAGCGTA
TTTTAGCAAACGTGAGTACGCACTACCTCCAGCTAGATACTATCATCAAG
AAACGCGAGATGGGATGATATATTCTGGTTACCTTACCAGACATAGTTAT
GCATCAACGGGAGACACGCGGATTGTAGT.

19. The method of detecting Bacillus anthracis of claim 1 wherein said at least one chromosomal DNA signature comprises:
(SEQ ID NO: 17)
CACGTGAGTTTACACCATACGCGGCGAACGCAAAGCGTTCAAGATTAGA
ACAAGAAGTGAATATTGATTTTTATAAAAGAGAAATTTTCACATATGCAG
ATGCTTGTATCGTCACAGTAAGGATTACAAATCCAGATGGCTCAACTGAG
TATCAAAAAGGAGAAATAAGTACAGAAAACATTGTATGTACTAATATCG
TATGGGGCGAGGATGAAGTTTCATTCGAAATGAGAGCAAGTGCTAG.

20. The method of detecting Bacillus anthracis of claim 1 wherein said at least one chromosomal DNA signature comprises:
(SEQ ID NO: 18)
GGAACAACTTTCCCACAAGCTGAACTTGAGTAGAAGCCAGATCAAAAAC
TGGGAAACTGATCGGTATCAGCCAGATATAGATACGTTGGTTATCATCGC
CTCCTTCTTCAATGTTTCGGTAGACGCGCTTATTGGGTTCACGAATGA

21. The method of detecting Bacillus anthracis of claim 1 wherein said at least one chromosomal DNA signature comprises:
(SEQ ID NO: 19)
AGGAACGTGCGACGACTAGCTGAAAAGCGTAGGGTGTAAGCCAATGAC
ATCCGAAATGGGGAGCATCTTATATAAAAGATGATGATATAGTCTGGTCT
GTATAGTGATATACAGAAGTTCATAAGAGAACTGGCAGGATGTTGCGAG
TCCTGTTGAACATATCGGGTG.

22. The method of detecting Bacillus anthracis of claim 1 wherein said at least one chromosomal DNA signature comprises:
(SEQ ID NO: 20)
TTTCTTATCCAACAGGTGTATGCTTATGTTTTATAAAAAAGTTTGATCAT
AGCTTATTTACAATTCTTTGATTAAAGAGATAAAGCATCGCACCTTTTTT
ATAAACGTCTTAACTTTATTTCAAAACTACACTATATTTACTAACCTACT
AATCTCCTACTCAACCGTAACAGACTTAGCTAAGTTACGTGGCTTATCAA
CGTCACACTCGC.

23. The method of detecting Bacillus anthracis of claim 1 wherein said at least one chromosomal DNA signature comprises:
(SEQ ID NO: 21)
TCAAACGTTGCCGAATAAGCCAATTCATTTAGATGCGTTACTTAATAAAC
TATTTTTAGGTAATACGAAGGACCGTATTAAAGCAGCAATTGAGTTAATT
GAGTGGGTGCTTCATAAATAATATTGTCTAATATTGTCGGTAAATCGATA
TGTTGTGTCGAATCGTC.

24. The method of detecting Bacillus anthracis of claim 1 wherein said at least one chromosomal DNA signature comprises:
(SEQ ID NO: 22)
TTTGGCATTGTGTTAGATGAGGGGTATTTCTATGATGAATTAACATTGAAA
GAAATGAAAAATGTGATTGCCCCTTCTTATACTGACTGGGATGAAGCAGT
CTTCTTATCTTATATTAAACAATTTAATTTAAATCTAAGACAAAAAATTTC
TACTCTATCTAAAGGGATGCGGATGAAATTTGCGGTTGCTTTGGCTTTATC
ACA.

25. The method of detecting Bacillus anthracis of claim 1 wherein said at least one chromosomal DNA signature comprises:
(SEQ ID NO: 23)
CAGAGCGTTTCTTAAATGAAGAGGATTTAATTGGAGAGCATTTAGAGAG
AGAAGATATTGATATCACATATTTAGATGACACGATGTATATATTGTTGC
CATATAAGAATGATCAAGAATTAAAACAGAAAAAGTTAGATAAAATCC
AAATTGAGGCATATGATGAA.

26. The method of detecting Bacillus anthracis of claim 1 wherein said at least one chromosomal DNA signature comprises:
(SEQ ID NO: 24)
AAAGGCGGTTTAGAATTTGGACCGTATCAAAATTTAAAACAAGTTGAGT
CGATAGCAGAAAAGCCAATTCAAGCATTTCAAGTTTTAAATACCATTCTT
GAGAAAATGGAAGAGAAAATGTTCTATATGAAGGAAAGACATTATACA
AACGTTGTAGAAACAAATATAAAAGAGCGTCATTTCATTATAGTTGATGA
AGGGGCTGAACTTTGTCCTGATAAAAGCATGGGTAAAGAGCAGCA.

27. The method of detecting Bacillus anthracis of claim 1 wherein said at least one chromosomal DNA signature comprises:
(SEQ ID NO: 25)
TGTAAATGAACGCCTTGACCAATCACCAGCAGGGAATTCAGTTTGTAGAT
TTTCTGTCCATGATCCGTTGATAAATTGGAATACAAGAAAAGTAACGTAA
ACATCATTTTGAAGATCGACACTGAATGTCATGTCTTTACCTTTTTCAAAA
TTACCCATCTTAGTATTATCTAAATGGAATTGATAGAAGGAGTCGG.

28. The method of detecting Bacillus anthracis of claim 1 wherein said at least one chromosomal DNA signature comprises:
(SEQ ID NO: 26)
AAACAATGTTGGATGGTAAATGGCATTACTTCCACGGCAAATTAAAAGTT
GTGGATGGGAAAGTGCGTGTTTACTTTGGGATGTTGAAGACGGGAAATGT
AAAAGGTGCTAAAACAAGAATAACCGGAGTGAAATTTGCGAATGG.

29. The method of detecting Bacillus anthracis of claim 1 wherein said at least one chromosomal DNA signature comprises:
(SEQ ID NO: 27)
CCGAGCTTTAATGGAAATGGAGAATTATTATATGATTTTAGGGATTTCAT
ATTTTAATATTTTTTGTATTGTAGTGATTATCTTGTTTTTCTTTATTTTT
TGGAGATTGCATGTATGGTATAACAAGAAACACAATGTTCCTAAGGTTTT
TCAATGGTTTCCTAGAAAATGGGGTAAAAGAAAAGTGTCAGAGCATTTAT
CACAATTGAATGAAAAGCTAGAGGCTGAAGGTAAAGG.

30. The method of detecting Bacillus anthracis of claim 1 wherein said at least one chromosomal DNA signature comprises:
(SEQ ID NO: 28)
GGAGAAACAGATAGTGGTGAAAGCTGGGAGTATATTTACTTCTTAGATG
AAATAAAGCATCAAGCCATTAGTTTAAGTGTCTTTAATAGATTATTAGAT
TATGAAGAAGGAAATCTAATACAAGGCTTTAGAGT.

31. A method of detecting Bacillus anthracis, comprising the steps of:
utilizing four separate loci on a Bacillus anthracis chromosome,
using a multiplex polymerase chain reaction analysis of the four separate loci on the Bacillus anthracis chromosome to produced a set of twenty eight chromosomal DNA signatures, and
using at least one of said twenty eight chromosomal DNA signatures to detect genetically Bacillus anthracis.

32. The method of detecting Bacillus anthracis of claim 31 wherein said step of using at least one of said twenty eight chromosomal DNA signatures to detect genetically Bacillus anthracis uses one or more of the following chromosomal DNA signatures:
(SEQ ID NO:1)
ACCTTTTGCTAAAGTGATTTCACCTTTTGCATTAATTTTTTCTGCA
GCTGGCGTTACAAATTCAATACCCTCTGGTGAAACAACTTCAGT,
or
(residues 3-157 of SEQ ID NO:2)
AAGTGGCAAAAGGGGAATTTAGAATATGCTAGTTTCCGTTCTATGA
CAATCGATGAATTTATTGATCCGTTGTCTTTTCTTAAAAAGATTGCTTCT
TTGTTTGAATTAGAAATTCAATATCGTGCTGGAGTTGTAGGCTCTCAAGT
TGTTGGGCG,
or
(SEQ ID NO:3)
TCCCAATACATATGAGCGATTCGCCTTTATAAACGACGTATTCCTT
TGAACTCGTTATGACACTCATTACTCAACTCCCCTTTTCTACTAAAATAG
CGTTTTTGTTTGGTTTTTTTCTTCACATAATCCGTCCTATTTGATTTTTA
CATACCACC,
or
(SEQ ID NO:4)
TTGGATCAGCGTTTCTGAATTCAGCTAATAATACAACGTCTTCTGG
ATCTAATGAATATAATCTTGTTAATGCAAGTGGAGTTGATATAAACGTAG
CACAGAAACTTAGGGGAATACGTAATTATAGCATTAAAGTAGATGGAAT
TGAGCGATATGGGGA,
or
(SEQ ID NO:5)
GGCCCCATAACTTGATTTCTCTTCAATATGAAAGTTGCTGCCTCAT
CAACATTTTCGAAAATAAAATCTTCATCATAAATCGTATGACAACCTTCC
CACCTTATAATTATAGGAAAGGCACCCGACGCCATTCCTTCAGCTGGAGC
AAGAT,
or
(SEQ ID NO:6)
GAAATGAATGCAGAAAAATTAGAGCTTGCGAAAAATCCACCTGAC
AGTGCAGCAGTACAAGAAATATTTGCTCAATTAGAAGGTTGGGGTAAAG
CTATTGCTAAAGGTGGAGAAGAACGTAAACAAGCATTTGTA,
or
(SEQ ID NO: 7)
AATTCTCATCCTACAGCATCTCCAATAATATATATTCCAACCAAA
AGATGACCATGTTACGGTTATAAAATACCAATCTATTATTTCATCGCGTC
CTTCACAACATCTTGAATCATAACGACTTCTGTTTTCATATCAACAGAGG
GTGCTTCTTCA,
or
(SEQ ID NO: 8)
GGTGGGTAAATGATGAGTGTTTCAACAAAAATTAATCAGCTAATA
AGTCTTAGAGAAAAAATCGCTAAAGATTTAGGGTTTTATGTTAACTTTGA
TTTAGCGAATTCAAGTTGGTCATACTCTAAAAAGGCTGGTCAAATCAAAA
A
or
(SEQ ID NO: 9)
TGTGGAAGAATCCACTGTCCAAGAACGTGAGCGTGTGCTATGGGA
TGAAATGGCTTCACCAATAGGATGGACTCCTGTTACTGGGCAAGTTGAGG
AAATGAAAGAGACAGA,
or
(SEQ ID NO: 10)
GAAGGACGATACAGACATTTATTGGGAACTACACATGCACTAGGG
TCATTGTTCATTAGCTCATCGATTGCATTATTAACAACGGCTTTTATTGT
CCTTACGCATTTATCTAGCTTTATGAACAATTACTATTTTTGGATAGGGC
TTGCTATTCAACTTGCGGT,
or
(SEQ ID NO: 11)
TCGCAAGATAAATGGGAAGACGAAAAGCGGGCTGGACAATATTC
TCCGACATACCAGGGACAGCTTCCAAGTGAAATGGTAAATGTGATTGGT
ACGGGAGGA,
or
(SEQ ID NO:12)
TTGGTAAAGTAGAAGCATCTTGGTTAAGAGCAGGATTACTCATGG
GTATGACGATAAAAACAAGCAATACAAATGAACATTTGCATATGGAAAA
CCAGGTGATGCGTTTTGTAAACCAAGGGTCAGATAAGATGG,
or
(SEQ ID NO:13)
ATATGTGGTTAGGAGCCAATGAAGGTGATGGTGACTTCTTTGTAC
ATAAACATACTTTACAAAAAAGCTTGTACAAGCCAGCTTCTGATTGGGAA
TGGTGGAGAGGAAAACGTCAGGTGATTATTGGACTCGATTTGT,
or
(SEQ ID NO: 14)
ACTACTCGACAAGGACGTTTGTATTTCCGGCTGTCAGTGTCGGCG
CGCCGCATCAAAGATACCGGACATTTATTGTTGGCCACTCCAACGACAAG
TCAAAATTACAAACCGATCCGAGAGTTGTGCCCTTCAGAAGCAAATGGGA
AACATGG,
or
(SEQ ID NO:15)
TGATATTTATGACCAAGATTCAATATACGAATATAATCCCAAAAG
AGCCAATCATGCAATGGAGTTCATTGAGAATTTTTGCAAGCATTCAAAGG
GGAAATCGGCTGGAAAACCTATTGACCTTGAGCTATGGC,
or
(SEQ ID NO:16)
CCAGGTCCATCCTATGTTGCAGTAGAAACGTATGTTACAGTAGAA
GCGTATTTTAGCAAACGTGAGTACGCACTACCTCCAGCTAGATACTATCA
TCAAGAAACGCGAGATGGGATGATATATTCTGGTTACCTTACCAGACATA
GTTATGCATCAACGGGAGACACGCGGATTGTAGT,
or
(SEQ ID NO:17)
CACGTGAGTTTACACCATACGCGGCGAACGCAAAGCGTTCAAGAT
TAGAACAAGAAGTGAATATTGATTTTTATAAAAGAGAAATTTTCACATAT
GCAGATGCTTGTATCGTCACAGTAAGGATTACAAATCCAGATGGCTCAAC
TGAGTATCAAAAAGGAGAAATAAGTACAGAAAACATTGTATGTACTAAT
ATCGTATGGGGCGAGGATGAAGTTTCATTCGAAATGAGAGCAAGTGCTA
C,
or
(SEQ ID NO:18)
GGAACAACTTTCCCACAAGCTGAACTTGAGTAGAAGCCAGATCAA
AAACTGGGAAACTGATCGGTATCAGCCAGATATAGATACGTTGGTTATC
ATCGCCTCCTTCTTCAATGTTTCGGTAGACGCGCTTATTGGGTTCACGAA
TGA,
or
(SEQ ID NO:19)
AGGAACGTGCGACGACTAGCTGAAAAGCGTAGGGTGTAAGCCAA
TGACATCCGAAATGGGGAGCATCTTATATAAAAGATGATGATATAGTCTG
GTCTGTATAGTGATATACAGAAGTTCATAAGAGAACTGGCAGGATGTTGC
CAGTCCTGTTGAACATATCGGGTG,
or
(SEQ ID NO:20)
TTTCTTATCCAACAGGTGTATGCTTATGTTTTATAAAAAAGTTT
TGATCATAGCTTATTTACAATTCTTTGATTAAAGAGATAAAGCATCGCAC
CTTTTTTATAAACGTCTTAACTTTATTTCAAAACTACACTATATTTACTA
ACCTACTAATCTCCTACTCAACCGTAACAGACTTAGCTAAGTTACGTGGC
TTATCAACGTCACACTCGC
or
(SEQ ID NO: 21)
TCAAACGTTGCCGAATAAGCCAATTCATTTAGATGCGTTACTTA
ATAAACTATTTTTAGGTAATACGAAGGACCGTATTAAAGCAGCAATTGAG
TTAATTGAGTGGGTGCTTCATAAATAATATTGTCTAATATTGTCGGTAAA
TCGATATGTTGTGTCGAATCGTC,
or
(SEQ ID NO:22)
TTGGCATTGTGTTAGATGAGGGGTATTTCTATGATGAATTAACA
TTGAAAGAAATGAAAAATGTGATTGCCCCTTCTTATACTGACTGGGATGA
AGCAGTCTTCTTATCTTATATTAAACAATTTAATTTAAATCTAAGACAAA
AAATTTCTACTCTATCTAAAGGGATGCGGATGAAATTTGCGGTTGCTTTG
GCTTTATCACA
or
(SEQ ID NO:23)
CAGAGCGTTTCTTAAATGAAGAGGATTTAATTGGAGAGCATTTAG
AGAGAGAAGATATTGATATCACATATTTAGATGACACGATGTATATATTG
TTGCCATATAAGAATGATCAAGAATTAAAACAGAAAAAGTTAGATAAAA
TCCAAATTGAGGCATATGATGAA,
or
(SEQ ID NO:24)
AAAGGCGGTTTAGAATTTGGACCGTATCAAAATTTAAAACAAGTT
GAGTCGATAGCAGAAAAGCCAATTCAAGCATTTCAAGTTTTAAATACCA
TTCTTGAGAAAATGGAAGACAAAATGTTCTATATGAAGGAAAGACATTA
TACAAACGTTGTAGAAACAAATATAAAAGAGCGTCATTTCATTATAGTTG
ATGAAGGGGCTGAACTTTGTCCTGATAAAAGCATGGGTAAAGAGCAGCA,
or
(SEQ ID NO:25)
TGTAAATGAACGCCTTGACCAATCACCAGCAGGGAATTCAGTTTGT
AGATTTTCTGTCCATGATCCGTTGATAAATTGGAATACAAGAAAAGTAAC
GTAAACATCATTTTGAAGATCGACACTGAATGTCATGTCTTTACCTTTTT
CAAAATTACCCATCTTAGTATTATCTAAATGGAATTGATAGAAGGAGTC
GG,
or
(SEQ ID NO:26)
AAACAATGTTGGATGGTAAATGGCATTACTTCCACGGCAAATTAA
AAGTTGTGGATGGGAAAGTGCGTGTTTACTTTGGGATGTTGAAGACGGGA
AATGTAAAAGGTGCTAAAACAAGAATAACCGGAGTGAAATTTGCGAATGG,
or
(SEQ ID NO:27)
CCGAGCTTTAATGGAAATGGAGAATTATTATATGATTTTAGGGATT
TCATATTTTAATATTTTTTGTATTGTAGTGATTATCTTGTTTTTCTTTA
TTTTTTGGAGATTGCATGTATGGTATAACAAGAAACACAATGTCCTAAG
GTTTTTCAATGGTTTCCTAGAAAATGGGGTAAAAGAAAAGTGTCAGAGC
ATTTATCACAATTGAATGAAAAGCTAGAGGCTGAAGGTAAAGG,
or
(SEQ ID NO:28)
GGAGAAACAGATAGTGGTGAAAGCTGGGAGTATATTTACTTCTTA
GATGAAATAAAGCATCAAGCCATTAGTTTAAGTGTCTTTAATAGATTATT
AGATTATGAAGAAGGAAATCTAATACAAGGCTTTAGAGT.
